(19) United States
(12) Patent Application Publication
(10) Pub. No.: US 2009/0196889 A1
(43) Pub. Date: Aug. 6, 2009

(54) CONTROLLED ABSORPTION OF STATINS IN THE INTESTINE

(75) Inventor: Adel Penhasi, Holon (IL)

Correspondence Address: WINSTON & STRAWN LLP;PATENT DEPARTMENT, 1700 K STREET, N.W., WASHINGTON, DC, 20006, US

(73) Assignee: DEXCEL PHARMA TECHNOLOGIES LTD.

(21) Appl. No.: 11/719,786

(22) PCT Filed: Nov. 22, 2005
(86) PCT No.: PCT/IL05/01234

§ 371 Date: Mar. 12, 2009

Related U.S. Application Data
(60) Provisional application No. 60/629,336, filed on Nov. 22, 2004.

(51) Int. Cl. A61K 9/00; A61K 9/28; A61K 31/351

(52) U.S. Cl. 424/400; 424/472; 514/460

(57) ABSTRACT

The present invention provides a controlled absorption formulation in which modified release of active ingredient preferentially occurs in the lower gastrointestinal tract, including the colon. The formulation supports a significantly higher bioavailability of the active ingredient into the body of the subject than can be achieved from the currently used conventional formulation, such that therapeutically significant plasma levels of statin are maintained for an extended period after administration. The formulation preferably features a core over which an outer coating is layered. The core is optionally and preferentially in the form of a tablet.

### FIELD OF THE INVENTION

The present invention relates to a formulation for the controlled absorption of a medication, and in particular, to a formulation for the delayed onset, modified release of HMG-CoA reductase inhibitors (statins) predominantly in the lower gastrointestinal (GI) tract.

### BACKGROUND OF THE INVENTION

Modified release formulations for oral administration of drugs are beneficial for a number of reasons. For example, they enable the patient to ingest the formulation less frequently, which may lead to increased patient compliance with the dosing regimen. They may also result in fewer side effects, as peaks and troughs of the level of the drug in the bloodstream of the patient may be decreased, leading to a more even drug level in the blood over a period of time. Such formulations may also provide a longer plateau concentration of the drug in the blood. The size and frequency of dosing is determined by the pharmacodynamic and pharmacokinetic properties of the drug. The slower the rate of absorption, the less the blood concentrations fluctuate within a dosing interval. This enables higher doses to be given less frequently. For drugs with relatively short half-lives, the use of modified-release products may maintain therapeutic concentrations over prolonged periods.

Currently, delayed onset, modified release drug delivery systems administered by the oral route are usually based on either a gel forming matrix or coated formulations, or the combination thereof.

A delayed onset drug delivery system should preferentially deliver drugs to any part of the lower GI tract, as a site for topical delivery and subsequent absorption of the drug. This concept relies on the fact that the retention time of the drug delivery system through the colon may be the longest as compared to other parts of gastrointestinal tract. Likewise, such a delivery system could also advantageously use the unique continuous absorption characterizing the colon, which results in flatter, more consistent concentration levels of the drug in blood. Such an absorption, of course, can contribute significantly to reduction of the fluctuations in blood drug concentration thus preventing the side effects which may appear upon using either immediate or conventional controlled release formulations, thereby improving compliance

Many different types of delayed onset formulations for delivery to the colon are known in the art. These include pH-dependent delivery systems; pH -independent delivery systems, including systems depending on factors such as hydrolytic degradation, hydrolysis, enzymatic degradation, and physical degradation, such as dissolution; and time-dependent delivery systems. Time-dependent systems release their drug load after a preprogrammed time delay. To attain colonic release, the lag time should equal the time taken for the system to reach the colon. The small intestinal transit time is generally considered to be in the region of three to four hours.

The statins are a class of compounds which contain a moiety that can exist as either a 3-hydroxy lactone ring or as the corresponding open ring dihydroxy acid. The structural formulas of these and additional HMG-CoA reductase inhibitors, are described in M. Yalpani, “Cholesterol Lowering Drugs”, Chemistry & Industry, pp. 85-89 (1996).

The statins are orally effective in the reduction of serum cholesterol levels, by competitively inhibiting 3-hydroxy-3-methylglutaryl coenzyme A (HMG CoA) reductase, and play an important role in primary and secondary prevention of ischemic heart disease and myocardial infarct.

The statins include natural fermentation products lovastatin (described in U.S. Pat. No. 4,231,938) and mevastatin (described in U.S. Pat. No. 3,671,523); as well as a variety of semi-synthetic and totally synthetic products, which include simvastatin (U.S. Pat. No. 4,444,784); pravastatin sodium salt (U.S. Pat. No. 4,346,227); fluvastatin sodium salt (U.S. Pat. No. 5,354,772); atorvastatin calcium salt (U.S. Pat. No. 5,273,995); and cerivastatin sodium salt (also known as rivastatin; U.S. Pat. No. 5,177,080).

An osmosis-controlled release formulation for a statin is taught in U.S. Pat. No. 5,916,595, to Andrx which comprises a core containing a water swellable polymer and an osmotic agent, a channeling agent and a water insoluble cellulose polymer. Water is drawn into the tablet, which expands to the point where the outer coating fails in one particular area to form a constricted opening which releases the internal contents of the tablet which contain the drug. Thereafter, the aqueous medium of the tablet shell continues to release the drug as it dissolves until the osmotic pressure inside the tablet shell equals that of the surrounding environment. At the late stages of the in vivo release, the tablet shell collapses and/or disintegrates completely in order to substantially release the remaining drug. Complete release occurs over a period of 4-30 h.

U.S. Pat. No. 5,882,682 to Merck teaches controlled delivery of simvastin from a core by use of a water insoluble coating which contains apertures. The release rate of the simvastatin is a function of the number and size of the apertures in the coating, and again is a slow, extended form of release.

U.S. Pat. No. 4,997,658 to Merck teaches a method for lowering plasma cholesterol by using a HMG-CoA reductase inhibitor in a sustained release manner over a period of 6-24 hours as a slow, extended form of release, thereby reducing the amount of HMG-CoA reductase inhibitor circulating in the bloodstream.

WO 01/34123 to Andrx teaches a controlled release dosage form for a drug which may include the statins, in which the release is gradual, and occurs at about 10 to about 32 hours after oral administration; again the drug emerges from the formulation in a slow, extended form of release. This dosage form is intended to provide a moderate level of plasma statin concentration, wherein the mean time to maximum plasma concentration of the drug is about 10 to 32 hours after oral administration. This application does not relate to the way by which a higher blood plasma concentration of the active material may be obtained after administration.

WO 04/021972 to Biovail discloses formulations which putatively decrease the concentration of lovastatin and simvastatin and their active metabolites in the systemic circulation and at the same time provide increased concentrations of these statins in the liver. The disclosure teaches extended release formulations as preferred over a burst release formulation, and the structure of the formulations taught may for example feature a number of compartments.

US Patent Application 2003/0176502 to Athapharma describes controlled-release formulations of pravastatin in the small intestine, thereby limiting systemic exposure of the body to pravastatin.

WO 01/32162 describes a method comprising administration of an HMG CoA reductase inhibitor in a slow-release formulation to the small intestine that provides a clinically effective level in the portal vein and liver, but less than that required to provide a clinically effective blood level in the peripheral circulation.

WO 00/33821 to BMS describes an enteric-coated pravastatin bead formulation. WO 98/15290 to Astra describes a sustained release formulation of fluvastatin. EP1036563 describes a delayed-release oral formulation of dihydroxy open acid statin.

A gastrointestinal controlled delivery system is disclosed in U.S. Pat. Nos. 5,840,332 and 6,703,044, neither of which relate to the use of those formulations for very poorly water soluble drugs in general and make no reference whatsoever to the statins in particular.

International Patent Application PCT/IL05/00539 of some of the applicants of the present invention teaches a delayed burst release oral formulation for localized release of a statin in the GI tract. That formulation comprises a core comprising a statin and a burst controlling agent and an outer coating comprising a water insoluble hydrophobic carrier and a water insoluble hydrophilic particulate matter. The particulate matter, which allows entry of liquid into the core, is preferably a hydrophilic yet water insoluble polymer.

Various references teach the metabolism and pharmacokinetics of statins in the human body (see for example M. J. Garcia et al., Clinical Pharmacokinetics of Statins, Clin. Pharmacol. 2003, 25 (6): 457-481).

Simvastatin is administered as the inactive lactone prodrug that must be hydrolyzed in the plasma and liver to the beta-hydroxy acid form for pharmacological activity. Simvastatin is believed to be metabolized in the liver and intestine, at least by the enzyme CYP3A, considering the beta-hydroxy acid form as the drug, the major active metabolites are 6-beta-hydroxymethyl and 6-beta-hydroxy simvastatin, which retain approximately 40% and 50%, respectively, of HMG-CoA reductase activity. Absorption reaches 60% while the bioavailability of the beta-hydroxy acid form following oral administration of simvastatin is less than 5%.

The poor bioavailability of simvastatin is mainly attributed to its low solubility in gastrointestinal fluids, low permeability through the mucosal membrane, and extensive first-pass metabolism. Since simvastatin (as stated above) is believed to be a CYP3A4 substrate, simvastatin may be expected to undergo significant intestinal metabolism.

The above cited reference also teaches that about 87% of the absorbed dose of simvastatin undergoes hepatic metabolism. The activation of simvastatin is by carboxyesterase-mediated hydrolysis, which occurs to a slight extent in plasma and in a higher extent in the liver. Both the parent lactone and the acid forms are normally present in very small amounts in the plasma, due to a high hepatic extraction ratio.

Simvastatin and its active acid forms are highly bound to plasma proteins, primarily to albumin (more than 95%). More than 98% of simvastatin is protein bound versus 94.5% for the open hydroxyl acid form. As only unbound drug is assumed to be able to enter the tissues, the high protein binding and low plasma concentrations of simvastatin are in agreement with the low peripheral tissue exposure in humans.

Physicians' Desk Reference 58th edition, 2004, pages 2113-211.8 teaches the metabolism, pharmacokinetics, pharmacodynamics and side effects of simvastatin, and is hereby incorporated by reference as if fully set forth herein.

### SUMMARY OF THE INVENTION

The background art does not teach or suggest a delayed onset, modified release formulation for delivery of statins to the GI tract including the lower GI tract and the colon, providing an increased blood concentration of a statin and/or active forms of said statin, relative to that resulting from the administration of an equivalent dose of conventional immediate release formulations.

Nor does the background teach or suggest a delayed onset, modified release formulation, which provides greater bioavailability. The background art also does not teach or suggest such a formulation, which provides fewer side effects, for greater patient compliance and comfort.

There remains an unmet need for formulations of statins with improved bioavailability and pharmacokinetics of the active species while minimizing side effects and reduced dosage.

The present invention overcomes the deficiencies of known formulations of statins by providing a controlled absorption formulation for once a day administration in which modified release of the active ingredient preferably occurs in the lower GI tract including the colon. Alternatively, such release may occur in the small intestine. The formulation provides significant plasma levels of a statin or its metabolites that are maintained for an extended period after administration.

Without wishing to be limited by a single hypothesis, the formulation of the present invention is believed to have preferential release of the drug in the lower GI tract, resulting in increased amount of a statin and its active hydroxyl acid forms than would have been formed if the drug were allowed to be absorbed into the bloodstream prior to reaching the appropriate section(s) of the intestine.

Local intestinal production of a greater amount of the active metabolite, probably through the activity of colonic natural flora, or via other metabolic routes, will further enhance the desired clinical effect and allow the achievement of intestinal drug levels of these metabolites that are unattainable by systemic or conventional oral delivery.

By using the formulation according to the present invention, which is preferably a modified release formulation, it may be possible to obtain increased production of active forms in the gut than that which can be obtained through carboxyesterase-mediated hydrolysis in the liver.

Further advantages of at least partial colonic delivery are that statins probably have greater solubility in the colon, and colon transit times are longer, resulting in increased time of exposure of the drug, and hence greater absorption.

Orally administered drugs or chemical agents that are processed to active forms in the intestinal environment can be administered to a patient who suffers from impaired liver function. Impaired liver function prevents or diminishes the normal hepatic metabolism of drugs to active metabolites. The increased production of active forms following administration of the formulations of the present invention is believed to reduce stress on the liver. The liver enzyme CYP3A4 is also present in the intestine, hence metabolism in the intestine can serve an alternative for metabolism in the liver for such drugs in these patients.

Another reason for delivering statin in the lower GI tract using the formulations of the present invention is to avoid high concentrations of CYP3A4, in which is largely present at a high concentration in the upper GI tract, and thereby to enable the release of statin to take place in the lower GI tract where the concentration of CYP3A4 is relatively poor. This process can increase the bioavailability of the statin.

A further reason for delivering statin in the lower GI tract using the formulations of the present invention is reduce the potential for interaction between drugs. This is in the light of the fact that many drugs may either induce or inhibit the activity of CYP3A4, and thus the bioavailability of statin may be affected.

One of the advantages of the present invention is that optionally a reduced dosage of a statin may be used in comparison to the presently available formulations, which may lead to the following beneficial effects:
1. Reduced liver side effects, such as a reduced level of transaminase for example (dose-related side effect).
2. Reduced incidence of rhabdomyolysis, muscle pain, and/or reduced level of CPK (dose-related side effect).
3. Reduced gastrointestinal effects including but not limited to nausea, dyspepsia, flatulence, and/or constipation (may be dose related side effects; however, the present invention is expected to be effective to reduce these side effects in any event, regardless of whether they are dose related).
4. Better tolerated multiple drug treatment in which at least one additional drug is metabolized by the liver.

A further advantage of the present invention is that a reduced food effect on the release may be obtained, since the formulation according to the present invention provides a release occurring predominantly in the lower gastrointestinal tract including the colon. Metabolism and absorption of orally administered drugs are commonly known to be affected by interactions with food. The formulation of the present invention is expected to be little affected or even unaffected by such interactions, since metabolism and absorption of the statin occurs in the intestine, optionally and preferably in the colon.

According to a first aspect, the formulation according to the present invention provides a drug delivery formulation for localized drug release of a statin in the gastrointestinal tract comprising a core, over which an outer coating is layered.

According to one embodiment, the core is preferably in the form of a tablet.

According to other embodiments, the core may be selected from the group consisting of pellets, microparticles, agglomerates, capsule or any other solid dosage form.

According to one embodiment the present invention provides a drug delivery formulation for localized drug release of a statin in the gastrointestinal tract comprising a core comprising at least one statin, wherein the core includes at least one release controlling agent and an outer coating over the core the outer coating comprising a polymer that erodes and/or is ruptured after a predetermined period of time post administration.

According to various alternative embodiments, the core is selected from the group consisting of a compressed tablet, pellets, microparticles, agglomerates, and capsules. According to various embodiments the statin is selected from lovastatin, mevastatin simvastatin, pravastatin, fluvastatin, atorvastatin, and cerivastatin also known as rivastatin, and salts thereof. The dosage levels of the active ingredient may easily be determined by one of ordinary skill in the art. According to certain currently preferred embodiments the statin is selected from simvastatin, atorvastatin and lovastatin.

According to a preferred embodiment of the present invention, the composition comprises a core containing a statin, a burst controlling agent and a disintegrant, the core being covered by a coating selected from the group consisting of a pH dependent coating film, preferably an enteric coating; a combination of at least one water soluble polymer and at least one water insoluble polymer; a combination of at least one swellable polymer and at least one water insoluble polymer; a combination of at least a water soluble pore forming agent and at least one water insoluble polymer; at least one swellable gel forming polymer; at least one erodible polymer; a combination of at least one pH dependent polymer and at least one water insoluble polymer; or a two-layer coating comprising a rupturable outer layer and swellable inner layer.

The burst controlling agent preferably comprises a water insoluble polymer for controlling the rate of penetration of water into the core and raising the internal pressure (osmotic pressure) inside the core. Such a burst controlling agent is preferably able to swell upon contact with liquid.

According to various embodiments, the water insoluble polymer is selected from the group consisting of cross-linked polysaccharide, water insoluble starch, microcrystalline cellulose, water insoluble cross-linked peptide, water insoluble cross-linked protein, water insoluble cross-linked gelatin, water insoluble cross-linked hydrolyzed gelatin, water insoluble cross-linked collagen modified cellulose, and cross-linked polyacrylic acid.

According to specific embodiments, the cross-linked polysaccharide is selected from the group consisting of insoluble metal salts or cross-linked derivatives of alginate, pectin, xanthan gum, guar gum, tragacanth gum, and locust bean gum, carrageenan, metal salts thereof, and covalently cross-linked derivatives thereof.

According to specific embodiments, the modified cellulose is selected from the group consisting of cross-linked derivatives of hydroxypropylcellulose, hydroxypropylmethylcellulose, hydroxyethylcellulose, methylcellulose, carboxymethylcellulose, and metal salts of carboxymethylcellulose.

According to certain currently preferred embodiments, the water insoluble polymer is calcium pectinate or microcrystalline cellulose.

According to specific embodiments, the disintegrant is selected from the group consisting of croscarmellose sodium, crospovidone (cross-linked PVP) sodium carboxymethyl starch (sodium starch glycolate), cross-inked sodium carboxymethyl cellulose (Croscarmellose), pregelatinized starch (starch 1500), microcrystalline starch, water insoluble starch, calcium carboxymethyl cellulose, magnesium aluminum silicate and a combination thereof. More preferably, the disintegrating agent is croscarmellose sodium. Some commercial superdisintegrants suitable for use in the present invention include, Ac-Di-Sol, Primojel, Explotab, and Crospovidone.

According to some embodiments, the core further comprises at least one of an absorption enhancer, a binder, a hardness enhancing agent, and another excipient. According to specific embodiments the binder is selected from the group consisting of Povidone (PVP: polyvinyl pyrrolidone), low molecular weight HPC (hydroxypropyl cellulose), low molecular weight BPMC (hydroxypropyl methylcellulose), low molecular weight carboxy methyl cellulose, ethylcellulose, gelatin polyethylene oxide, acacia, dextrin, magnesium aluminum silicate, starch, and polymethacrylates. Optionally and preferably, the core also includes a stabilizer. More preferably, the stabilizer comprises at least one or more of butyl hydroxyanisole, ascorbic acid and citric acid.

According to some embodiments, the core further comprises a wicking agent Preferably, the wicking agent is selected from the group consisting of colloidal silicon dioxide, kaolin, titanium dioxide, fumed silicon dioxide, alumina, niacinamide, sodium lauryl sulfate, low molecular weight polyvinyl pyrrolidone, m-pyrol, bentonite, magnesium aluminum silicate, polyester, polyethylene, or mixtures thereof.

According to some embodiments, the core further comprises a filler. Preferably, the filler is selected from the group consisting of microcrystalline cellulose, starch, lactitol, lactose, a suitable inorganic calcium salt, sucrose, or a combination thereof. More preferably the filler is lactose monohydrate.

According to preferred embodiments of the present invention, the core further comprises an antioxidant. Preferably, the antioxidant is selected from the group consisting of 4,4 (2,3 dimethyl tetramethylene dipyrochatechol), Tocopherol-rich extract (natural vitamin E), α-tocopherol (synthetic Vitamin E), β-tocopherol, γ-tocopherol, δ-tocopherol, Butylhydroxinon, Butyl hydroxyanisole (BHA), Butyl hydroxytoluene (BHT), Propyl Gallate, Octyl gallate, Dodecyl Gallate, Tertiary butylhydroquinone (TBHQ), Fumaric acid, Malic acid, Ascorbic acid (Vitamin C), Sodium ascorbate, Calcium ascorbate, Potassium ascorbate, Ascorbyl palmitate, Ascorbyl stearate, Citric acid, Sodium lactate, Potassium lactate, Calcium lactate, Magnesium lactate, Anoxomer, Erythorbic acid, Sodium erythorbate, Erythorbin acid, Sodium erythorbin, Ethoxyquin, Glycine, Gum guaiac, Sodium citrates (monosodium citrate, disodium citrate, trisodium citrate), Potassium citrates (monopotassium citrate, tripotassium citrate), Lecithin, Polyphosphate, Tartaric acid, Sodium tartrates (monosodium tartrate, disodium tartrate), Potassium tartrates (monopotassium tartrate, dipotassium tartrate), Sodium potassium tartrate, Phosphoric acid, Sodium phosphates (monosodium phosphate, disodium phosphate, trisodium phosphate), Potassium phosphates (monopotassium phosphate, dipotassium phosphate, tripotassium phosphate), Calcium disodium ethylene diamine tetra-acetate (Calcium disodium EDTA), Lactic acid, Trihydroxy butyrophenone and Thiodipropionic acid.

According to a preferred embodiment, the core further comprises ascorbic acid, which has several hydroxyl and/or carboxylic acid groups, and is able to provide a supply of hydrogen for regeneration of the primary antioxidant, exerting a synergistic effect on the inactivated antioxidant free radical.

According to a currently most preferred embodiment, the primary antioxidant is BHA.

According to preferred embodiments of the present invention, the core further comprises a chelating agent. Preferably, the chelating agent is selected from the group consisting of Antioxidants, Dipotassium edentate, Disodium edentate, Edetate calcium disodium, Edetic acid, Fumaric acid, Malic acid, Maltol, Sodium edentate, Trisodium edetate.

According to some embodiments of the present invention, the core further comprises one or both of a chelator and a synergistic agent (sequestrate). Preferably, the sequestrate is selected from the group consisting of citric acid and ascorbic acid. Without wishing to be limited by a single hypothesis, chelating agents and sequestrates may optionally be differentiated as follows. A chelating agent, such as citric acid, is intended to help in chelation of trace quantities of metals thereby assisting to prevent the loss of the active ingredient(s), such as simvastatin, by oxidation. A sequestrate, such as ascorbic acid, optionally and preferably has several hydroxyl and/or carboxylic acid groups, which can provide a supply of hydrogen for regeneration of the inactivated antioxidant free radical. A sequestrate therefore preferably acts as a supplier of hydrogen for rejuvenation of the primary antioxidant. Therefore, the combination of both a chelator and a sequestrate is preferred to protect the active statin ingredient.

According to additional embodiments, the core further comprises a flow regulating agent. Preferably, the flow regulating agent includes at least one of colloidal silicon dioxide and aluminum silicate. Most preferably, the flow regulating agent is colloidal silicon dioxide.

Preferably, the core further comprises a lubricant. More preferably, the lubricant is selected from the group consisting of stearate salts; stearic acid, corola oil, glyceryl palmitostearate, hydrogenated vegetable oil, magnesium oxide, mineral oil, poloxamer, polyethylene glycole, polyvinyl alchol, sodium benzoate, talc, sodium stearyl fumarate, compritol (glycerol behenate), and sodium lauryl sulfate (SLS) or a combination thereof. Most preferably, the lubricant is magnesium stearate.

Optionally, the outer coating further comprises a plasticizer. More preferably, the plasticizer includes at least one of dibutyl sebacate, polyethylene glycol and polypropylene glycol, dibutyl phthalate, diethyl phthalate, triethyl citrate, tributyl citrate, acetylated monoglyceride, acetyl tributyl citrate, triacetin, dimethyl phthalate, benzyl benzoate, butyl and/or glycol esters of fatty acids, refined mineral oils, oleic acid, castor oil, corn oil, camphor, glycerol and sorbitol or a combination thereof.

Optionally, the outer coating further comprises a stiffening agent. More preferably, the stiffening agent is cetyl alcohol.

Optionally, the outer coating or the core or both further comprises at least one of a wetting agent, a suspending agent, and a dispersing agent, or a combination thereof. More preferably, the wetting agent is selected from the group consisting of poloxamer, polyoxyethylene ethers, polyoxyethylene sorbitan fatty acid esters (polysorbates), polyoxymethylene stearate, sodium lauryl sulfate, sorbitan fatty acid esters, benzalkonium chloride, polyethoxylated castor oil, and docusate sodium. Also more preferably, the suspending agent is selected from the group consisting of alginic acid, bentonite, carbomer, carboxymethylcellulose, carboxymethylcellulose calcium, hydroxyethylcellulose, hydroxypropyl cellulose, microcrystalline cellulose, colloidal silicon dioxide, dextrin, gelatin, guar gum, xanthan gum, kaolin, magnesium aluminum silicate, maltitol, medium chain triglycerides, methylcellulose, polyoxyethylene sorbitan fatty acid esters (polysorbates), povidone (PVP), propylene glycol alginate, sodium alginate, sorbitan fatty acid esters, and tragacanth. Most preferably, the dispersing agent is selected from the group consisting of poloxamer, polyoxyethylene sorbitan fatty acid esters (polysorbates) and sorbitan fatty acid esters.

Optionally, the formulation may comprise an enteric coating disposed on the outer coating. The enteric coating is more preferably selected from the group consisting of cellulose acetate phthalate, hydroxy propyl methyl cellulose acetate succinate, poly(methacrylic acid, methyl methacrylate)1:1 and (Eudragite® L100), poly(methacrylic acid, ethyl acrylate):1 (Eudragite® L30D-55).

According to optional but preferred embodiments of the present invention, the coating comprises a combination of at least one water soluble polymer and at least one water insoluble polymer. Optionally and preferably, the water-soluble polymer is selected from the group consisting of polyvinyl alcohol, polyvinylpyrrolidone (PVP), methylcellulose, hydroxypropylcellulose, hydroxypropylmethyl cellulose, or polyethylene glycol, carboxymethyl cellulose (sodium salt), hydroxyethyl cellulose, a water soluble gum, polysaccharide and/or mixtures thereof.

Optionally and preferably, the water insoluble polymer is selected from the group consisting of a podimethylaminoethylacrylate/ethylmethacrylate copolymer, an ethylmethacrylate/chlorotrimethylammoniumethyl methacrylate copolymer, a dimethylaminoethylmethacrylate/methylmethacrylate and butylmethacrylate copolymer, a copolymer based on neutral methacrylic acid esters and dimethylaminoethyl methacrylate esters, an ethylacrylate and methylacrylate/ethylmethacrylate and methyl methylacrylate copolymer, ethylcellulose, shellac, zein, and waxes, paraffin, cellulose acetate, cellulose propionate, cellulose acetate propionate, cellulose acetate butyrate, cellulose acetate phthalate, cellulose triacetate, poly(methyl methacrylate), poly(ethylmethacrylate), poly (butyl methacrylate), poly(isobutyl methacrylate), and poly(hexyl methacrylate), poly (isodecyl methacrylate), poly(lauryl methacrylate), poly(phenyl methacrylate), poly (methylacrylate), poly(isopropyl acrylate), poly(isobutyl acrylate) poly(octadecyl acrylate), poly(ethylene), poly(ethylene) low density, poly(ethylene) high density, poly(ethylene oxide), poly(ethyleneterephthalate), poly(vinyl isobutyl ether), poly(vinyl acetate), poly(vinyl chloride) and polyurethane, and/or mixtures thereof.

More preferably, the water insoluble polymer is ethylcellulose.

According to optional but preferred embodiments of the present invention, the coating comprises a combination of at least a water soluble pore forming agent and at least one water insoluble polymer. Optionally and preferably, the pore-forming agent is selected from the group consisting of saccharose, sodium chloride, potassium chloride, polyvinylpyrrolidone, and/or polyethyleneglycol, water soluble organic acids, sugars and sugar alcohol. Optionally, the pore forming compound is distributed uniformly throughout said water insoluble polymer. Alternatively, the pore forming compound is distributed randomly throughout said water insoluble polymer. Optionally, the pore-forming compound comprises about 1 part to about 35 parts for each about 1 to about 10 parts of said water insoluble polymer.

According to optional but preferred embodiments of the present invention, the coating comprises an erodible polymer. Optionally and preferably the erodible composition comprises at least one of a slow dissolving and a slow disintegrating composition. Preferably, the erodible composition comprises at least one of a slowly water soluble polymer and a swellable polymer. Also preferably, the erodible composition further comprises a disintegrant.

According to optional but preferred embodiments of the present invention, the coating comprises at least one swellable gel-forming polymer. Preferably, the swellable gel-forming polymer is selected from the group consisting of cellulosic polymers; vinyl polymers; acrylic polymers and copolymers, methacrylic acid copolymers, ethyl acrylate-methyl methacrylate copolymers, natural and synthetic gums, gelatin, collagen, proteins, polysaccharides, pectin, pectic acid, alginic acid, sodium alginate, polyaminoacids, polyalcohols, polyglycols; and mixtures thereof.

More preferably, the cellulosic polymer is selected from the group consisting of methylcellulose, carboxymethylcellulose, hydroxypropylcellulose, hydroxypropylmethylcellulose, and hydroxyethylcellulose. Most preferably, the cellulosic polymer comprises hydroxymethylcellulose.

Optionally and preferably, the coating comprises a water insoluble polymer that is swellable, although alternatively it may be non swellable.

According to optional but preferred embodiments of the present invention, the coating further comprises at least one of a lubricant, a flow promoting agent, a plasticizer, an antisticking agent, natural and synthetic flavorings and natural- and synthetic colorants.

Preferably, the lubricant further comprises at least one of polyethylene glycol, polyvinylpyrrolidone, talc, magnesium stearate, glyceryl behenate, stearic acid, and titanium dioxide.

According to optional but preferred embodiments of the present invention, the coating comprises a combination of at least one swellable polymer and at least one water insoluble polymer.

According to optional but preferred embodiments of the present invention, the coating comprises a combination of at least one pH dependent polymer and at least one water insoluble polymer.

According to optional but preferred embodiments of the present invention, the coating comprises a two-layer coating comprising a rupturable outer layer and swellable inner layer. Preferably, the two-layer coating ruptures independently of said core. Optionally and preferably, the inner layer comprises a disintegrant.

Preferably, the inner layer comprises at least one polymer being able to swell when contacted by water. More preferably, the at least one polymer is selected from the group consisting of hydroxypropylmethyl cellulose, high molecular weight of carboxymethyl cellulose, high molecular weight of hydroxypropyl cellulose, high molecular weight of hydroxyethyl cellulose, high molecular weight of hydroxymethyl cellulose, polyhydroxyethyl methacrylate, polyhydroxymethyl methacrylate, polyacrylic acid, carbopol, polycarbophil, gums, polysaccharides, modified polysaccharides, cross-linked polysaccharide, water insoluble starch, microcrystalline cellulose, water insoluble cross-linked peptide, water insoluble cross-linked protein, water insoluble cross-linked gelatin, water insoluble cross-linked hydrolyzed gelatin, water insoluble cross-linked collagen modified cellulose, and cross-linked polyacrylic acid.

Most preferably, the cross-inked polysaccharide is selected from the group consisting of insoluble metal salts or cross-linked derivatives of alginate, pectin, xanthan gum, guar gum, tragacanth gum, and locust bean gum, carrageenan, metal salts thereof, and covalently-cross-linked derivatives thereof.

Also most preferably, the modified cellulose is selected from the group consisting of cross-linked derivatives of hydroxypropylcellulose, hydroxypropylmethylcellulose, hydroxyethylcellulose, methylcellulose, carboxymethylcellulose, and metal salts of carboxymethylcellulose.

According to optional but preferred embodiments of the present invention, the inner layer comprises a disintegrant embedded in a water soluble film forming polymer.

According to optional but preferred embodiments of the present invention, the inner layer comprises a combination of a water soluble polymer forming a film matrix, and a swellable water insoluble polymer particulate embedded into said film matrix.

According to optional but preferred embodiments of the present invention, the rupturable outer layer comprises a brittle polymer.

According to optional but preferred embodiments of the present invention, the rupturable outer layer comprises at least one permeation-enhancing agent.

According to optional but preferred embodiments of the present invention, the rupturable outer layer comprises a water insoluble polymer selected from the group consisting of a dimethylaminoethylacrylate/ethylmethacrylate copolymer, the copolymer being based on acrylic and methacrylic acid esters with a low content of quaternary ammonium groups, wherein the molar ratio of the ammonium groups to the remaining neutral (meth)acrylic acid esters is approximately 1:20, the polymer corresponding to USP/NF “Ammonio Methacrylate Copolymer Type A”, an ethylmethacrylate/chlorotrimethylammoniumethyl methacrylate copolymer, the copolymer based on acrylic and methacrylic acid esters with a low content of quaternary ammonium groups wherein the molar ratio of the ammonium groups to the remaining neutral (meth)acrylic acid esters is 1:40, the polymer corresponding to USP/NF “Ammonio Methacrylate Copolymer Type B”, a dimethylaminoethylmethacrylate/methylmethacrylate and butylmethacrylate copolymer, a copolymer based on neutral methacrylic acid esters and dimethylaminoethyl methacrylate esters wherein the polymer is cationic in the presence of acids, an ethylacrylate and methylacrylate/ethylmethacrylate and methyl methylacrylate copolymer, the copolymer being a neutral copolymer based on neutral methacrylic acid and acrylic acid esters, ethylcellulose, shellac, zein, and waxes.

Preferably, the water insoluble polymer comprises ethylcellulose.

In one embodiment, the in vivo blood plasma concentration of the statin and/or a pharmaceutically acceptable salt and/or ester thereof is controlled by a lag time, providing a controlled absorption of the statin and/or a pharmaceutically acceptable salt and/or ester thereof and/or related active forms. In one specific embodiment, the formulations of the present invention are characterized in that the in vivo blood plasma concentration of the statin or a pharmaceutically acceptable salt or ester thereof in the subject is substantially zero for at least about 1.5 hours after oral administration of the formulation. In another specific embodiment, the formulations of the present invention are characterized in that the in vivo blood plasma concentration of the statin or a pharmaceutically acceptable salt or ester thereof in the subject is substantially zero for at least about two hours after oral administration of the formulation. In another specific embodiment, the in vivo blood plasma concentration of the statin or a pharmaceutically acceptable salt or ester thereof in the subject is substantially zero for at least about three hours after oral administration of the formulation. In yet another specific embodiment, the in vivo blood plasma concentration of the statin or a pharmaceutically acceptable salt or ester thereof in the subject is substantially zero for at least about four hours after oral administration of the formulation. The term “substantially zero”, as used herein, means that the statin is either not detected in the blood, or only minor amounts of the statin are detected in the blood.

According to one embodiment, the delayed burst release formulation of the present invention provides an increased amount of a statin, a pharmaceutically acceptable salt or ester thereof, or an active form thereof to the circulation of a subject, compared to a substantially similar dose of a conventional immediate release formulation of the stain. As used herein, the term “substantially similar dose” means a dose which is either equivalent or is substantially similar, for example a difference of not more than about 25%. The term “increased amount” means that administration of the formulations of the present invention result in higher blood levels of the statins or their active metabolites (e.g., 10% higher, 20% higher, 50% higher 100% higher, 200% higher, 500% higher etc.), as compared with blood levels achieved by administration of conventional statin formulations. The levels of the statins can be measured by determining the plasma concentration of the statins as a function of time following administration of the formulation, as known to a person of skill in the art. As demonstrated herein, administration of several simvastatin and pitavastatin formulations according to the present invention to subjects resulted in blood levels that were significantly higher than the blood levels achieved after administration of conventional formulations of these statins. Further, importantly, the blood levels were maintained for significantly longer time periods as compared with the conventional formulation. For example, blood levels can be maintained for at least about 6 hours, preferably for about 8 hours, about 10 hours, about 12 hours and most preferably for about 24 hours after the delayed burst release occurs.

According to an alternative embodiment, the delayed burst release formulation of the present invention provides enhanced bioavailability of a statin, a pharmaceutically acceptable salt or ester thereof, or an active form thereof in a subject, compared to a substantially similar dose of an immediate release formulation of the stain. The term “enhanced bioavailability” means that administration of the formulations of the present invention results in higher bioavailability of the statins or their active metabolites (e.g., 10% higher, 20% higher, 50% higher 100% higher, 200% higher, 500% higher etc.), as compared with the bioavailability achieved by administration of conventional statin formulations. Bioavailability can be measured for example by comparing the AUC values obtained after administration of the formulations, as known to a person of skill in the art. As demonstrated herein, administration of several simvastatin and pitavastatin formulations according to the present invention to subjects resulted in AUC values that were more than two fold higher than the AUC values obtained after administration of conventional formulations of these statins. Further, the AUC values were maintained for significantly longer time periods as compared with the conventional formulation, for example for at least about 6 hours, preferably for about 8 hours, about 10 hours, about 12 hours and most preferably for about 24 hours after the delayed burst release occurs.

According to yet another alternative embodiment, the delayed burst release formulation of the present invention provides a therapeutically effective amount of a statin, a pharmaceutically acceptable salt or ester thereof, or an active form thereof into the circulation of a subject. The term “therapeutically effective amount” refers to an amount of the statin which will result in a therapeutic effect of the disease or condition being treated, for example high blood cholesterol.

The present invention represents an improvement over WO 2004/021972 to Biovail, as the Biovail application seeks to reduce the concentration of statins in the blood circulation. In contrast, the present invention provides an increased concentration of statins or active forms thereof in the blood circulation relative to the dose administered, thus resulting in the administration of relatively lower dose of a statin or active forms thereof in the formulation administered to the subject (patient), thereby potentially reducing side effects by decreasing the total dose of statin to which the body of the subject is exposed.

As explained above, the statins are a class of compounds which contain a moiety that can exist as either a 3-hydroxy lactone ring or as the corresponding open ring dihydroxy acid. Typically, the statins can be administered as the inactive lactone prodrugs that must be hydrolyzed in the plasma and liver to the beta-hydroxy acid form for pharmacological activity. In accordance with the present invention, the delayed burst release formulations described herein are capable of providing a therapeutically effective amount of the hydroxy acid metabolite of a statin or a pharmaceutically acceptable salt or ester thereof into the circulation of a subject.

According to other preferred embodiments of the present invention, there is provided a formulation for administering a statin to a subject, featuring a relatively lower dose of said statin. By “relatively lower dose” it is meant a dose that provides at least the same or similar pharmaceutical and/or therapeutic effect (if not a greater effect) as a conventional dose of a statin, while featuring a lower amount of statin than the conventional dose of the statin. It should be noted that a similar principle may optionally be applied for dosage forms featuring a plurality of different statins.

The core of the formulations of the present invention contains a statin, which is preferably selected from simvastatin, lovastatin, mevastatin, pravastatin, fluvastatin, atorvastatin, cerivastatin and pitavastatin or pharmaceutically acceptable salts, esters, metabolites, hydrates, polymorphs, or crystals thereof. According to one currently preferred embodiment the statin is simvastatin. According to another currently preferred embodiment the statin is pitavastatin. According to other preferred embodiments the statin is lovastatin or atorvastatin.

The term “statin” as used herein includes also pharmaceutically acceptable salts, esters, metabolites, hydrates, polymorphs, or crystals thereof, and includes both statins in the lactone form or in the corresponding open dihydroxy acid.

The term “simvastatin” includes simvastatin and pharmaceutically acceptable salts, esters, metabolites, hydrates, polymorphs, or crystals thereof, in the lactone form or in the corresponding open dihydroxy acid, as disclosed for example in U.S. Pat. No. 4,444,784, which is hereby incorporated by reference in its entirety as if fully set forth herein.

The term “lovastatin” includes lovastatin and pharmaceutically acceptable salts, esters, metabolites, hydrates, polymorphs, or crystals thereof, in the lactone form or in the corresponding open dihydroxy acid, as disclosed for example in U.S. Pat. No. 4,231,938, which is hereby incorporated by reference in its entirety as if fully set forth herein.

The term “mevastatin” includes mevastatin and pharmaceutically acceptable salts, esters, metabolites, hydrates, polymorphs, or crystals thereof, in the lactone form or in the corresponding open dihydroxy acid, as disclosed for example in U.S. Pat. No. 3,671,523, which is hereby incorporated by reference in its entirety as if fully set forth herein.

The term “pravastatin” includes pravastatin and pharmaceutically acceptable salts, esters, metabolites, hydrates, polymorphs, or crystals thereof, in the lactone form or in the corresponding open dihydroxy acid, as disclosed for example in U.S. Pat. No. 4,346,227, which is hereby incorporated by reference in its entirety as if fully set forth herein.

The term “fluvastatin” includes fluvastatin and pharmaceutically acceptable salts, esters, metabolites, hydrates, polymorphs, or crystals thereof, in the lactone form or in the corresponding open dihydroxy acid, as disclosed for example in U.S. Pat. No. 5,354,772, which is hereby incorporated by reference in its entirety as if fully set forth herein.

The term “atorvastatin” includes atorvastatin and pharmaceutically acceptable salts, esters, metabolites, hydrates, polymorphs, or crystals thereof, in the lactone form or in the corresponding open dihydroxy acid, as disclosed for example in U.S. Pat. No. 5,273,995, which is hereby incorporated by reference in its entirety as if fully set forth herein.

The term “rivastatin” includes rivastatin and pharmaceutically acceptable salts, esters, metabolites, hydrates, polymorphs, or crystals thereof, in the lactone form or in the corresponding open dihydroxy acid, as disclosed for example in U.S. Pat. No. 5,177,080, which is hereby incorporated by reference in its entirety as if fully set forth herein.

The term “pitavastatin” (“nisvastatin”) includes pitavastatin and pharmaceutically acceptable salts, esters, metabolites, hydrates, polymorphs, or crystals thereof, in the lactone form or in the corresponding open dihydroxy acid, as disclosed for example in U.S. Pat. No. 5,011,930, U.S. Pat. No. 5,872,130, U.S. Pat. No. 5,856,336, which are hereby incorporated by reference in their entirety as if fully set forth herein.

As used herein, the term “active form” refers to any form of a molecule that can function as an HMG-CoA reductase inhibitor including the active ingredient administered and any active derivative resulting from metabolism or otherwise obtained from the parent molecule that can act as an HMG-CoA reductase. For example in the case of simavastatin marketed under the tradename ZOCOR® the known active forms include α-hydroxyacid of simvastatin and its 6β-hydroxy, 6β-hydroxymethyl, and 6β-exomethylene derivatives. The term “metabolite”, as used herein, includes any active form of the statin as described herein.

Suitable pharmaceutically acceptable salts include but are not limited to inorganic salts such as, for example, sodium, potassium, ammonium, calcium, and the like.

The doses of the statins to be used in the formulations of the present invention can be determined by a person of skill in the art, and will vary depending on the statin being used, the patient, and the condition being treated. Typical known therapeutic doses for each of the statins can be used as a guide to determine the appropriate dose to be used herein. As mentioned above, the formulations of the present invention preferably contain a reduced dose of the statin, as compared with the corresponding conventional formulation, preferably up to about 60% of the conventional dose for each statin.

### BRIEF DESCRIPTION OF THE DRAWINGS

The invention is herein described, by way of example only, with reference to the accompanying drawings, wherein:

FIG. 1 shows the in-vitro dissolution profile for the different coating formulation examples according to the present invention which were coated with Kollidone and ethyl cellulose (Ethocel);

FIG. 2 shows the in-vitro dissolution profile from the core coated with HPMC/Ethocel;

FIG. 3 shows the results of dissolution for a formulation according to the present invention with enterically coated cores; and

FIG. 4 shows Simvastatin release (%) from tablets coated with inner swelling layer and outer water permeable layer.

### DESCRIPTION OF THE PREFERRED EMBODIMENTS

The present invention provides a formulation for controlled absorption of a statin, adapted so as to provide a time-delayed, modified release in the colon or small intestine. The formulation supports a lag time between oral administration and release of the active ingredient, providing higher bioavailability and lower dosage as compared to the currently used formulation. The formulation of the present invention optionally features non pH-dependent release, although alternatively and preferably features pH-dependent release, as for example with an enteric film coat. The term “statin” includes also pharmaceutically acceptable salts or esters thereof.

The term “modified release” preferably includes delayed burst release and optionally includes any type of delayed release.

The delivery system of the present invention provides a modified formulation comprising a statin for controlled delivery of the active ingredient to the gastrointestinal tract. The delivery system comprises a drug containing core surrounded by a coating that limits the access of liquid to the core thereby controlling the release of the drug from the core to the GI tract.

The formulation is optionally in the form of a coated tablet. Alternatively, the formulation may be in the form of a pellet, microparticles, agglomerate, capsule or any other solid dosage form.

The combination of the selected materials for the core and outer layer, and the relative concentrations thereof, as well as the thickness of the core matrix and outer layer, determine both the lag time, which is the time, post administration, when the release starts, as well as the rate of release of the drug.

### Burst Core Release

An optional but preferred embodiment according to the present invention wherein the modified release core is preferably a burst release core. Without wishing to be limited by a single hypothesis, a preferred embodiment of the formulation according to the present invention preferably features a core which contains a swellable material, covered by a coating through which water enters the core. The swellable material in the core then swells and bursts the coating, after which the core more preferably disintegrates slowly or otherwise releases the active ingredient. Another optional but preferred embodiment relates to a fast disintegrating core.

Release of the active agent of the present formulation preferably occurs within about 2-6 hours of oral administration, with a slightly longer delay occurring with the enteric coated embodiment.

This optional embodiment of a formulation of the present invention therefore provides a delayed onset, rapid burst release formulation for delivery of statins in the lower GI tract preferentially to the colon or small intestine, which provides higher blood levels of statin or its metabolites in the bloodstream in comparison to a conventional immediate release formulation. The bioavailability is shown to be higher than that of a known reference product. The formulations according to the present invention should result in fewer side effects, greater safety, efficacy, and patient compliance.

This optional embodiment of a formulation of the present invention preferably includes a burst-controlling agent, such that release occurs rapidly, within a period of less than 8 hours following oral administration, with selective absorption of the active agent in the lower GI tract.

In one embodiment the delayed burst release formulation is based on a fast disintegrating core. The core can be based on either a swellable non hydrogel forming formulation or non swellable non hydrogel forming formulation, but in any case it is preferably a fast disintegrating formulation. The swellable or non swellable components thereto may optionally be water insoluble polymers as described herein, but alternatively may comprise one or more of osmotic pressure-creating agents such as water soluble salts (low molecular weight) and water soluble polymers such as polyvinyl pyrrolidone, carboxymethyl cellulose, hydroxyethyl cellulose, hyroxymethyl cellulose, hydroxypropyl cellulose.

Such a formulation can prevent release of the active ingredient in the stomach and even in the upper GI tract for a predetermined period of time, for example up to about 2 hours, more preferably up to about 3 to 4 hours, most preferably up to about 6 hours, after which the release can take place in a burst manner (fast release). The core according to such an embodiment may comprise the active ingredient, a disintegrant and a burst controlling agent which is preferably a water swellable non hydrogel forming polymer, in which the core is preferably formed as a compressed tablet. More preferably, the core is in the form of one of a tablet, pellets, microparticles, agglomerate, and capsule.

The core may comprise the active ingredient, a filler and a disintegrant, or alternatively the active ingredient and one or more disintegrants.

More preferably, the burst controlling agent comprises a water insoluble polymer. Most preferably, the water insoluble polymer is selected from the group consisting of cross-linked polysaccharide, water insoluble starch, microcrystalline cellulose, water insoluble cross-linked peptide, water insoluble cross-linked protein, water insoluble cross-linked gelatin, water insoluble cross-linked hydrolyzed gelatin, water insoluble cross-linked collagen modified cellulose, and cross-linked polyacrylic acid.

Preferably, the cross-linked polysaccharide is selected from the group consisting of insoluble metal salts or cross-linked derivatives of alginate, pectin, xantham gum, guar gum, tragacanth gum, and locust bean gum, carrageenan, metal salts thereof, and covalently cross-linked derivatives thereof.

Preferably, the modified cellulose is selected from the group consisting of cross-linked derivatives of hydroxypropylcellulose, hydroxypropylmethylcellulose, hydroxyethylcellulose, methylcellulose, carboxymethylcellulose, and metal salts of carboxymethylcellulose.

Also most preferably, the water insoluble polymer is calcium pectinate or microcrystalline cellulose.

Optionally and preferably, the disintegrant is selected from the group consisting of croscarmellose sodium, crospovidone (cross-linked polyvinyl pyrolidone) sodium carboxymethyl starch (sodium starch glycolate), cross-linked sodium carboxymethyl cellulose (Croscarmellose), pregelatinized starch (starch 1500), microcrystalline starch, water insoluble starch, calcium carboxymethyl cellulose, magnesium aluminum silicate and a combination thereof. More preferably, the disintegrating agent is croscarmellose sodium.

The mechanism of disintegration is optionally and preferably based on swelling, wicking, and deformation of the disintegrants. Some commercial superdisintegrants for use in the present invention include, Ac-Di-Sol, Primojel, Explotab, and Crospovidone.

Preferably, the core further comprises at least one of an absorption enhancer, a binder, a hardness enhancing agent, and another excipient. More preferably, the binder is selected from the group consisting of Povidone (PVP: polyvinyl pyrrolidone), low molecular weight HPC (hydroxypropyl cellulose), low molecular weight HPMC (hydroxypropyl methylcellulose), low molecular weight carboxy methyl cellulose, ethylcellulose, gelatin polyethylene oxide, acacia, dextrin, magnesium aluminum silicate, starch, and polymethacrylates. Optionally and preferably, the core also includes a stabilizer. More preferably, the stabilizer comprises at least one of butyl hydroxyanisole, ascorbic acid and citric acid.

The core of the present invention optionally and preferably includes a wicking agent in addition to or as an alternative to a disintegrant. Wicking agents such as those materials already mentioned as disintegrants (e.g. microcrystalline cellulose) may be included if necessary to enhance the speed of water uptake. Other materials suitable for acting as wicking agents include, but are not limited to, colloidal silicon dioxide, kaolin, titanium dioxide, fumed silicon dioxide, alumina, niacinamide, sodium lauryl sulfate, low molecular weight polyvinyl pyrrolidone, m-pyrol, bentonite, magnesium aluminum silicate, polyester, polyethylene, mixtures thereof, and the like.

Alternatively or additionally, the core further comprises a filler. Preferably, the filler is selected from the group consisting of microcrystalline cellulose, starch, lactitol, lactose, a suitable inorganic calcium salt, sucrose, or a combination thereof. More preferably the filler is lactose monohydrate.

More preferably, the core further includes a chelating agent to increase chelation of trace quantities of metals thereby helping in preventing the loss of a statin such as Simvastatin by oxidation. Most preferably, the chelating agent is citric acid.

According to preferred embodiments of the present invention, the core further comprises a synergistic agent (sequestrate). Preferably, the sequestrate is selected from the group consisting of citric acid and ascorbic acid.

Without wishing to be limited by a single hypothesis, chelating agents and sequestrates may optionally be differentiated as follows. A chelating agent, such as (preferably) citric acid is intended to help in chelation of trace quantities of metals thereby assisting to prevent the loss of the active ingredient(s), such as a statin such as Simvastatin for example, by oxidation.

A sequestrate such as (preferably) ascorbic acid, optionally and preferably has several hydroxyl and/or carboxylic acid groups, which can provide a supply of hydrogen for regeneration of the inactivated Butyl hydroxyanisole (BHA) antioxidant free radical. A sequestrate therefore preferably acts as a supplier of hydrogen for rejuvenation of the primary antioxidant.

According to preferred embodiments of the present invention, the core further comprises an antioxidant. Preferably, the antioxidant is selected from the group consisting of 4,4 (2,3 dimethyl tetramethylene dipyrochatechol), Tocopherol-rich extract (natural vitamin E), α-tocopherol (synthetic Vitamin E), β-tocopherol, γ-tocopherol, δ-tocopherol, Butylhydroxinon, Butyl hydroxyanisole (BHA), Butyl hydroxytoluene (BHT), Propyl Gallate, Octyl gallate, Dodecyl Gallate, Tertiary butylhydroquinone (TBHQ), Fumaric acid, Malic acid, Ascorbic acid (Vitamin C), Sodium ascorbate, Calcium ascorbate, Potassium ascorbate, Ascorbyl palmitate, Ascorbyl stearate, Citric acid, Sodium lactate, Potassium lactate, Calcium lactate, Magnesium lactate, Anoxomer, Erythorbic acid, Sodium erythorbate, Erythorbin acid, Sodium erythorbin, Ethoxyquin, Glycine, Gum guaiac, Sodium citrates (monosodium citrate, disodium citrate, trisodium citrate), Potassium citrates (monopotassium citrate, tripotassium citrate), Lecithin, Polyphosphate, Tartaric acid, Sodium tartrates (monosodium tartrate, disodium tartrate), Potassium tartrates (monopotassium tartrate, dipotassium tartrate), Sodium potassium tartrate, Phosphoric acid, Sodium phosphates (monosodium phosphate, disodium phosphate, trisodium phosphate), Potassium phosphates (monopotassium phosphate, dipotassium phosphate, tripotassium phosphate), Calcium disodium ethylene diamine tetra-acetate (Calcium disodium EDTA), Lactic acid, Trihydroxy butyrophenone and Thiodipropionic acid.

More preferably, the core further comprises ascorbic acid, which has several hydroxyl and/or carboxylic acid groups, and is able to provide a supply of hydrogen for regeneration of the primary antioxidant, exerting a synergistic effect on the inactivated antioxidant free radical. Most preferably, the primary antioxidant is BHA. According to preferred embodiments of the present invention, the core further comprises a chelating agent. Preferably, the chelating agent is selected from the group consisting of Antioxidants, Dipotassium edentate, Disodium edentate, Edetate calcium disodium, Edetic acid, Fumaric acid, Malic acid, Maltol, Sodium edentate, Trisodium edetate. Also alternatively or additionally, the core further comprises a flow regulating agent. Preferably, the flow regulating agent includes at least one of colloidal silicon dioxide and aluminum silicate.

Most preferably, the flow regulating agent is colloidal silicon dioxide. Preferably, the core further comprises a lubricant. More preferably, the lubricant is selected from the group consisting of stearate salts; stearic acid, corola oil, glyceryl palmitostearate, hydrogenated vegetable oil, magnesium oxide, mineral oil, poloxamer, polyethylene glycole, polyvinyl alcohol, sodium benzoate, talc, sodium stearyl fumarate, compritol (glycerol behenate), and sodium lauryl sulfate (SLS) or a combination thereof. Most preferably, the lubricant is magnesium stearate.

### Outer Coating

The coating is selected from the group consisting of a pH dependent coating film (featuring a pH dependent polymer), preferably an enteric coating; a combination of at least one water soluble polymer and at least one water insoluble polymer; a combination of at least one swellable polymer and at least one water insoluble polymer; a combination of at least a water soluble pore forming agent and at least one water insoluble polymer; at least one swellable gel forming polymer; at least one erodible polymer; a combination of at least one pH dependent polymer and at least one water insoluble polymer; or a two-layer coating comprising a rupturable outer layer and swellable inner layer. These coatings are preferred embodiments of coatings for the present invention since, without wishing to be limited by a single hypothesis, they are structured so as to provide a delayed burst release in combination with a suitable core. These coatings are capable either of disintegration or of partial or complete loss of integrity, thereby supporting rapid release of material after disintegration of the core. Preferably, the core is a rapidly disintegrating core, and its rapid disintegration is supported by these coatings.

Optionally and preferably, the water insoluble polymer is hydrophobic and hence does not form a hydrogel.

According to this embodiment of the present invention, the pH dependent polymer of the outer coating is selected from the group consisting of a hydroxypropylmethyl cellulose phthalate, polyvinyl acetate phthalate, cellulose acetate phthalate, hydroxypropylmethyl cellulose acetate succinate, poly(methacrylic acid, methyl methacrylate)1:1 and poly(methacrylic acid, ethyl acrylate)1:1, alginic acid, and sodium alginate. A suitable enteric coating can be from Eudragit® polymers series (available from Rohm Pharma) which are polymeric lacquer substances based on acrylates and/or methacrylates. Suitable polymers which are slightly permeable to water, and exhibit a pH-dependent permeability include, but are not limited to, Eudragit® L, and Eudragit® S. Eudragit® L is an anionic polymer synthesized from methacrylic acid and methacrylic acid methyl ester. It is insoluble in acids and pure water. It becomes soluble in neutral to weakly alkaline conditions. The permeability of Eudragit® L is pH dependent. Above pH 5.0, the polymer becomes increasingly permeable.

An illustrative, non-limiting example of such a formulation is as follows. The formulation optionally and preferably comprises a pH dependent film coat, the polymeric material comprises methacrylic acid co-polymers, ammonio methacrylate co-polymers, or a mixture thereof. Methacrylic acid co-polymers such as Eudragit® S and Eudragit® L (Rohm Pharma) are suitable for use in the delayed onset, modified, release formulations of the present invention, these polymers are gastro-resistant and entero-soluble polymers, providing a delay in onset of the release depending on the pH, the type of the polymer (Eudragit® L or Eudragit® S) and the thickness of the film coat.

The films of Methacrylic acid co-polymers such as Eudragit® S and Eudragit® L are insoluble in pure water and diluted acids. They dissolve at higher pH values, depending on their content of carboxylic acid. Eudragit® S and Eudragit® L can be used as single components in the coating of the formulation of the present invention or in combination in any ratio. By using a combination of the polymers, the polymeric material may exhibit a solubility at a pH between the pHs at which Eudragit® L and Eudragit® S are separately soluble.

Optionally, the outer coating further comprises a plasticizer. More preferably, the plasticizer includes at least one of dibutyl sebacate, polyethylene glycol and polypropylene glycol, dibutyl phthalate, diethyl phthalate, triethyl citrate, tributyl citrate, acetylated monoglyceride, acetyl tributyl citrate, triacetin, dimethyl phthalate, benzyl benzoate, butyl and/or glycol esters of fatty acids, refined mineral oils, oleic acid, castor oil, corn oil, camphor, glycerol and sorbitol or a combination thereof.

In another embodiment according to the present invention the delayed onset, modified release formulation may comprise a fast disintegrating core formulation, as described above, and an outer coating, optionally comprising a combination of a water soluble polymer and/or a water swellable hydrophilic polymer and a water insoluble polymer. In this manner, where the film coating formulation features a combination of at least a water soluble polymer and at least a water insoluble polymer, it is possible to provide a delay time prior to the release of the active material, depending on the relative content (weight fraction) of the water soluble polymer in the outer coating, the thickness of the outer film coat, and the nature of the polymers present in the outer film coat. Without wishing to be limited by a single hypothesis, upon exposure of the formulation to the gastrointestinal fluids, the water soluble polymer starts to dissolve, leaving channels that allow penetration of the gastrointestinal fluids into the core, which may eventually lead to a relatively fast disintegration of the core and thus a burst release of the active material.

Another non-limiting, illustrative example of a suitable coating may be based on a core which can be formulated as described above for the previous embodiment, and an outer coating comprising a totally water soluble polymer and a water insoluble polymer. Suitable water-soluble polymers include, but are not limited to, polyvinyl alcohol, polyvinylpyrrolidone (PVP), methylcellulose, hydroxypropylcellulose, hydroxypropylmethyl cellulose, or polyethylene glycol, carboxymethyl cellulose (sodium salt), hydroxyethyl cellulose, a water soluble gum, polysaccharide and/or mixtures thereof.

Suitable water insoluble polymers of the outer coating are selected from the group consisting of a podimethylaminoethylacrylate/ethylmethacrylate copolymer, the copolymer being based on acrylic and methacrylic acid esters with a low content of quaternary ammonium groups, wherein the molar ratio of the ammonium groups to the remaining neutral (meth)acrylic acid esters is approximately 1:20, the polymer corresponding to USP/NF “Ammonio Methacrylate Copolymer Type A”, an ethylmethacrylate/chlorotrimethylammoniumethyl methacrylate copolymer, the copolymer based on acrylic and methacrylic acid esters with a low content of quaternary ammonium groups wherein the molar ratio of the ammonium groups to the remaining neutral (meth)acrylic acid esters is 1:40, the polymer corresponding to USP/NF “Ammonio Methacrylate Copolymer Type B”, a dimethylaminoethylmethacrylate/methylmethacrylate and butylmethacrylate copolymer, a copolymer based on neutral methacrylic acid esters and dimethylaminoethyl methacrylate esters wherein the polymer is cationic in the presence of acids, an ethylacrylate and methylacrylate/ethylmethacrylate and methyl methylacrylate copolymer, the copolymer being a neutral copolymer based on neutral methacrylic acid and acrylic acid esters, ethylcellulose, shellac, zein, and waxes, paraffin, cellulose acetate, cellulose propionate, cellulose acetate propionate, cellulose acetate butyrate, cellulose acetate phthalate, cellulose triacetate, poly(methyl methacrylate), poly(ethylmethacrylate), poly(butyl methacrylate), poly(isobutyl methacrylate), and poly(hexyl methacrylate), poly (isodecyl methacrylate), poly(lauryl methacrylate), poly(phenyl methacrylate), poly (methylacrylate), poly(isopropyl acrylate), poly(isobutyl acrylate) poly(octadecyl acrylate), poly(ethylene), poly(ethylene) low density, poly(ethylene) high density, poly(ethylene oxide), poly(ethyleneterephthalate), poly(vinyl isobutyl ether), poly(vinyl acetate), poly(vinyl chloride) and polyurethane, and/or mixtures thereof. More preferably, the water insoluble polymer is ethylcellulose.

An optional but preferred embodiment of such a coating comprises ethylcellulose (water insoluble polymer) and a copolymer of polyvinyl pyrrolidone and vinyl acetate (water soluble polymer). Preferably, the water insoluble polymer is present in an amount ranging from about 20% to about 95%, and the water soluble polymer is present in an amount ranging from about 5% to about 45% of the coating. More preferably, the coating further comprises a glidant. Most preferably, the glidant comprises Sieved Talc.

Optionally, the formulation may further comprise an enteric coating disposed on the outer coating.

Another non-limiting illustrative example of a coating may optionally feature an outer coating comprising a combination of a water swellable hydrophilic polymer and a water insoluble film-forming polymer. The swellable polymer may be a gel-forming polymer. This enables the swellable polymer participating in the outer film coat composition to be free of the requirement to fully dissolve. Since the swelling process of the swellable polymer in the outer film coat composition controls the diffusion process of the GI fluid through the film coat into the core, without wishing to be limited by a single hypothesis it is expected that it will be the only key factor for controlling the lag time. Another factor controlling the lag time is the thickness of the outer film coat.

Suitable swellable polymers typically interact with water and/or gastrointestinal fluids, which causes them to swell or expand to an equilibrium state. Acceptable polymers exhibit the ability to swell in water and/or gastrointestinal fluids, retaining a significant portion of such imbibed fluids within their polymeric structure. The polymers may swell or expand, usually exhibiting a 2- to 50-fold volume increase. The polymers can be non-cross-linked or cross-linked. The swellable hydrophilic polymer is responsible for introducing the gastrointestinal fluids into the core, leading to swelling of the core and eventually release of the active material, optionally through bursting of the core. The swellable polymers are hydrophilic polymers. Suitable polymers include, but are not limited to, poly(hydrox alkyl methacrylate) having a molecular weight of from 30,000 to 5,000.000; kappa-carrageenan; polyvinylpyrrolidone having a molecular weight of from 10,000 to 360,000; anionic and cationic hydrogels; polyelectrolyte complexes; poly(vinyl alcohol) having low amounts of acetate, cross-linked with glyoxal, formaldehyde, or glutaraldehyde and having a degree of polymerization from 200 to 30,000; a mixture including methyl cellulose, cross-linked agar and carboxymethyl cellulose; a water-insoluble, water-swellable copolymer produced by forming a dispersion of finely divided maleic anhydride with styrene, ethylene, propylene, butylene or isobutylene; water-swellable polymers of N-vinyl lactams; polysaccharide, water swellable gums, high viscosity of hydroxylpropylmethyl cellulose and/or mixtures of any of the foregoing.

The outer film coat may also optionally include a material that improves the processing of the polymers. Such materials are generally referred to as plasticizers and include, for example, adipates, azelates, benzoates, citrates isoebucates, phthalates, sebacates, stearates and glycols. Representative plasticizers include acetylated monoglycerides, butyl phthalyl butyl glycolate, dibutyl tartrate, diethyl phthalate, dimethyl phthalate, ethyl phthalyl ethyl glycolate, glycerin, ethylene glycol, propylene glycol, triacetin citrate, triacetin, tripropinoin, diacetin, dibutyl phthalate, acetyl monoglyceride, polyethylene glycols, castor oil, triethyl citrate, polyhydric alcohols, acetate esters, glycerol triacetate, acetyl triethyl citrate, dibenzyl phthalate, dihexyl phthalate, butyl octyl phthalate, diisononyl phthalate, butyl octyl phthalate, dioctyl azelate, epoxidised tallate, triisoctyl trimellitate, diethylhexyl phthalate, di-n-octyl phthalate, di-1-octyl phthalate, di-1-decyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, dibutyl sebacate, glyceryl monocaprylate, and glyceryl monocaprate. In one embodiment, the plasticizer is dibutyl sebacate. The amount of plasticizer used in the polymeric material typically ranges from about 10% to about 50%, for example, about 10, 20, 30, 40 or 50%, based on the weight of the dry polymer.

An optional but preferred embodiment of the above coating features a coating in which the swellable polymer comprises hydroxypropyl methyl cellulose (HPMC) and the water insoluble polymer comprises Ethyl cellulose. Preferably, the water insoluble polymer is present in an amount ranging from about 20% to about 95%, and the swellable polymer is present in an amount ranging from about 5% to about 45% of the coating. More preferably, the coating further comprises a surfactant. Most preferably, the surfactant comprises sodium lauryl sulphate. More preferably, the coating further comprises a stiffening agent. Most preferably, the stiffening agent comprises cetyl alcohol. More preferably, the coating further comprises a glidant. Most preferably, the glidant comprises sieved talc.

Optionally, the formulation may comprise an enteric coating disposed on the outer coating.

In another embodiment, the outer film coat comprises one or more water-insoluble film-forming polymers and one or more water-soluble pore-forming compounds. Suitable water-soluble pore-forming compounds include, but are not limited to, saccharose, sodium chloride, potassium chloride, polyvinylpyrrolidone, and/or polyethyleneglycol, water soluble organic acids, sugars and sugar alcohol. The pore-forming compounds may be uniformly or randomly distributed throughout the water insoluble polymer. Typically, the pore-forming compounds comprise about 1 part to about 35 parts for each about 1 to about 10 parts of the water insoluble polymers. The amount and particle size of pore-forming agent in the film coat, and the thickness of the outer film coat are expected to be the main parameters controlling the lag time. Optionally, the formulation may comprise an enteric coating disposed on the outer coating.

In another embodiment a delayed onset, modified release formulation based on a dry compress coating process may be considered. Such a dosage form may optionally feature a rapidly disintegrating core coated with an erodible composition using a double compress tabletation. Such an erodible composition may optionally feature slow dissolving or slow disintegrating pharmaceutically acceptable excipients such as, but not limited to, water soluble polymers that solubilize slowly, swellable polymer or a composition comprising a water soluble polymer that solubilizes slowly with a disintegrant or a swellable polymer with disintegrant. Alternatively the coating process can be carried out using a conventional coating process such as spraying of an erodible or swellable polymer. Such a solution may optionally include additional excipients like a disintegrant and talc.

When an erodible polymer is used, the erosion rate of such a coating may determine the lag time, therefore, the type of polymer being used as erodible polymer, may be expected to control the erosion rate of the coating can determine the lag time. Another parameter that can control the lag time is the amount of erodible polymer constituting the thickness of the coating.

When a swellable polymer is used, the coating layer, which typically comprises a hydrophilic gelling polymer or swellable polymer, swells on contact with gastro-intestinal juices to form a continuous film surrounding the core. The coating layer must sufficiently protect the integrity of the core for the desired period of time, without regard to the pH of the medium to which it is subjected. Once the desired, pre-delivery time period has elapsed, the core should be capable of relatively fast disintegration so that the statin is released in a burst manner at the predetermined delivery time.

The polymeric coating layer may comprise any suitable hydrophilic gelling polymer known to those skilled in the art. For example, suitable hydrophilic gelling polymers include but are not limited to cellulosic polymers, such as methylcellulose, carboxymethylcellulose, hydroxypropylcellulose, hydroxypropylmethylcellulose, hydroxyethylcellulose, and the like; vinyl polymers, such as polyvinylpyrrolidone, polyvinyl alcohol, and the like; acrylic polymers and copolymers, such as acrylic acid polymer, carbopol, methacrylic acid copolymers, ethyl acrylate-methyl methacrylate copolymers, natural and synthetic gums, such as guar gum, arabic gum, xanthan gum, gelatin, collagen, proteins, polysaccharides, such as pectin, pectic acid, alginic acid, sodium alginate, polyaminoacids, polyalcohols, polyglycols; and the like; and mixtures thereof. The preferred swellable polymeric coating layer comprises carbopol. The more preferred swellable polymeric coating layer comprises hydroxypropylmethylcellulose.

Alternatively, the swellable polymeric coating layer comprises other substances which are capable of becoming freely permeable with exactly defined kinetics following hydration in aqueous fluids. Such substances include but are not limited to saccharose, sorbitol, mannaese, and jaluronic acid; and the like.

In addition to the foregoing, the swellable polymeric coating layer may also include additional excipients such as lubricants, flow promoting agents, plasticizers, antisticking agents, natural and synthetic flavorings and natural and synthetic colorants. Specific examples of additional excipients include polyethylene glycol, polyvinylpyrrolidone, talc, magnesium stearate, glyceryl behenate, stearic acid, and titanium dioxide.

The swellable polymeric coating layer may be applied to the core using conventional film (or spray) coating techniques, double press coating or by the method involving the alternate application of binder and powdered polymeric coating particles.

In certain embodiments, gums for use in the compression coating include, for example and without limitation, heteropolysaccharides such as xanthan gum(s), homopolysaccharides such as locust bean gum, galactans, mannans, vegetable gums such as alginates, gum karaya, pectin, agar, tragacanth, accacia, carrageenan, tragacanth, chitosan, agar, alginic acid, other polysaccharide gums (e.g. hydrocolloids), and mixtures of any of the foregoing. Further examples of specific gums which may be useful in the compression coatings of the invention include but are not limited to acacia catechu, salai guggal, indian bodellum, copaiba gum, asafetida, cambi gum, Enterolobium cyclocarpum, mastic gum, benzoin gum, sandarac, gambier gum, butea frondosa (Flame of Forest Gum), myrrh, konjak mannan, guar gum, welan gum, gellan gum, tara gum, locust bean gum, carageenan gum, glucomannan, galactan gum, sodium alginate, tragacanth, chitosan, xanthan gum, deacetylated xanthan gum, pectin, sodium polypectate, gluten, karaya gum, tamarind gum, ghatti gum, Accaroid/Yacca/Red gum, dammar gum, juniper gum, ester gum, ipil-ipil seed gum, gum talha (acacia seyal), and cultured plant cell gums including those of the plants of the genera: acacia, actinidia, aptenia, carbobrotus, chickorium, cucumis, glycine, hibiscus, hordeum, letuca, lycopersicon, malus, medicago, mesembryanthemum, oryza, panicum, phalaris, phleum, poliathus, polycarbophil, sida, solanum, trifolium, trigonella, Afzelia africana seed gum, Treculia africana gum, detarium gum, cassia gum, carob gum, Prosopis africana gum, Colocassia esulenta gum, Hakea gibbosa gum, khaya gum, scleroglucan, zea, mixtures of any of the foregoing, and the like.

In certain especially preferred embodiments, the compression coating comprises a heteropolysaccharide such as xanthan gum, a homopolysaccharide such as locust bean gum, or a mixture of one or more hetero- and one or more homopolysaccharide(s). Heterodisperse excipients, previously disclosed as a sustained release tablet matrix in U.S. Pat. No. 4,994,276, U.S. Pat. No. 5,128,143, and U.S. Pat. No. 5,135,757, may be utilized in the compression coatings of the present invention. For example, in certain embodiments of the present invention, a gelling agent of both hetero- and homo-polysaccharides which exhibit synergism, e.g., the combination of two or more polysaccharide gums producing a higher viscosity and faster hydration than that which would be expected by either of the gums alone, the resultant gel being faster-forming and more rigid, may be used in the compression coatings of the present invention.

The term “heteropolysaccharide” as used in the present invention is defined as a water-soluble polysaccharide containing two or more kinds of sugar units, the heteropolysaccharide having a branched or helical configuration, and having excellent water-wicking properties and immense thickening properties.

An especially preferred heteropolysaccharide is xanthan gum, which is a high molecular weight (>10.sup.6) heteropolysaccharide. Other preferred heteropolysaccharides include derivatives of xanthan gum, such as deacylated xanthan gum, the carboxymethyl ether, and the propylene glycol ester.

The homopolysaccharide materials used in the present invention that are capable of cross-linking with the heteropolysaccharide include the galactomannans, i.e., polysaccharides that are composed solely of mannose and galactose. A possible mechanism for the interaction between the galactomannan and the heteropolysaccharide involves the interaction between the helical regions of the heteropolysaccharide and the unsubstituted mannose regions of the galactomannan. Galactomannans that have higher proportions of unsubstituted mannose regions have been found to achieve more interaction with the heteropolysaccharide. Hence, locust bean gum, which has a higher ratio of mannose to galactose, is especially preferred as compared to other galactomannans, such as guar and hydroxypropyl guar.

An additional embodiment comprises a tablet system featuring a fast disintegrating core, which is not necessarily swellable, coated with two distinct layers of swelling and rupturable coating layers, preferably comprising a rupturable outer layer and swellable inner layer in the two-layer coating. The rapidly disintegrating core containing statin is preferably coated sequentially with an inner swelling layer preferably containing superdisintegrant and an outer rupturable layer preferably comprising a brittle polymer. The latter coating layer may optionally include at least one permeation-enhancing agent in order to promote the diffusion of water into the rupturable coating layer. The swelling coating layer is responsible for bursting the outer coating layer (rupturable). This takes place when the swelling layer comes into the contact with water, where an internal force is exerted as a result of the swelling of this layer.

Such a coating has unique properties in that it is able to burst (split) independently of the core. The swellable inner layer is composed of a polymer or a combination of polymers being able to swell when contacted by water. The resulting osmotic pressure created from swelling can exert force on the rupturable outer layer to cause it to lose its integrity and eventually to burst. The swelling layer may be composed of a disintegrant embedded in a water soluble film forming polymer. Non-limiting examples of the polymers which can be utilized in the swellable inner layer are hydroxypropylmethyl cellulose, high molecular weight of carboxymethyl cellulose, high molecular weight of hydroxypropyl cellulose, high molecular weight of hydroxyethyl cellulose, high molecular weight of hydroxymethyl cellulose, polyhydroxyethyl methacrylate, polyhydroxymethyl methacrylate, polyacrylic acid, carbopole, polycarbophil, gums, polysaccharides, modified polysaccharides, cross-linked polysaccharide, water insoluble starch, microcrystalline cellulose, water insoluble cross-linked peptide, water insoluble cross-linked protein, water insoluble cross-linked gelatin, water insoluble cross-linked hydrolyzed gelatin, water insoluble cross-linked collagen modified cellulose, and cross-linked polyacrylic acid. According to specific embodiments, the cross-linked polysaccharide is selected from the group consisting of insoluble metal salts or cross-linked derivatives of alginate, pectin, xanthan gum, guar gum, tragacanth gum, and locust bean gum, carrageenan, metal salts thereof, and covalently cross-linked derivatives thereof. According to specific embodiments, the modified cellulose is selected from the group consisting of cross-linked derivatives of hydroxypropylcellulose, hydroxypropylmethylcellulose, hydroxyethylcellulose, methylcellulose, carboxymethylcellulose, and metal salts of carboxymethylcellulose. The swellable inner layer can be also based on combination of a water soluble polymer and a swellable water insoluble polymer particulate which is embedded into the water soluble polymer film matrix.

The rupturable outer layer is a water insoluble polymer which can be selected from the group consisting of a dimethylaminoethylacrylate/ethylmethacrylate copolymer, the copolymer being based on acrylic and methacrylic acid esters with a low content of quaternary ammonium groups, wherein the molar ratio of the ammonium groups to the remaining neutral (meth)acrylic acid esters is approximately 1:20, the polymer corresponding to USP/NF “Ammonio Methacrylate Copolymer Type A”, an ethylmethacrylate/chlorotrimethylammoniumethyl methacrylate copolymer, the copolymer based on acrylic and methacrylic acid esters with a low content of quaternary ammonium groups wherein the molar ratio of the ammonium groups to the remaining neutral (meth)acrylic acid esters is 1:40, the polymer corresponding to USP/NF “Ammonio Methacrylate Copolymer Type B”, a dimethylaminoethylmethacrylate/methylmethacrylate and butylmethacrylate copolymer, a copolymer based on neutral methacrylic acid esters and dimethylaminoethyl methacrylate esters wherein the polymer is cationic in the presence of acids, an ethylacrylate and methylacrylate/ethylmethacrylate and methyl methylacrylate copolymer, the copolymer being a neutral copolymer based on neutral methacrylic acid and acrylic acid esters, ethylcellulose, shellac, zein, and waxes. More preferably, the water insoluble polymer is ethylcellulose.

According to an optional but preferred embodiment of the present invention, there is provided a coating comprising an enteric coating. Preferably, the enteric coating comprises Hydroxypropylmethyl cellulose acetate succinate (HPMC AS).

More preferably, HPMC AS is present in an amount ranging from about 25% to about 90% of the enteric coating. Optionally and more preferably, the coating comprises a plasticizer. Most preferably, the plasticizer comprises triethyl citrate. Also optionally and more preferably, the coating comprises a surfactant. Most preferably, the surfactant comprises sodium lauryl sulfate.

### Therapeutic Uses

The formulations of the present invention are capable of providing a therapeutically effective amount of a statin, a pharmaceutically acceptable salt or ester thereof or an active form thereof to a subject, for an extended period of time after the burst release occurs. The formulations according to the present invention have increased efficacy and provide at least a similar, if not greater, pharmaceutical effect with the active ingredient, using a significantly decreased dosage amount as compared with conventional formulations known in the art regarding reduce of elevated total cholesterol, low density lipoprotein cholesterol, apolipoprotein B, triglycerides and increase of high density lipoprotein cholesterol. Preferably, the formulations of the present invention contain the statin in an amount that is up to about 60% as compared to an immediate release formulation, yet provides at least similar pharmaceutical efficacy. Thus, the novel formulations of the present invention are more effective than conventional statin formulations currently in use, and can be used for treating high cholesterol, ischemic heart disease and myocardial infarction, or any other disease or condition for which statins are indicated. The formulations of the present invention may even lead to new indications for the use of delayed burst release of simvastatin and can be used in new populations of patients in which the conventional statin formulations are not used for at present. The formulations of the present invention preferably comprise at least one statin in a decreased dosage amount of up to about 50% as compared to an immediate release formulation of the statin, while providing a substantially equivalent effect of lowering of LDL as a full dosage of the immediate release formulation.

Thus in one aspect, the present invention relates to a method for providing a therapeutically effective amount of a statin, a pharmaceutically acceptable salt or ester thereof or an active form thereof to a subject, comprising orally administering to the subject a modified release formulation as described herein, featuring a slowly disintegrating core, wherein the formulation releases substantially no statin in vitro for at least about 2 hours to about 6 hours, preferably at least about 2 hours, more preferably at least about 3 hours, also more preferably at least about 4 hours, also more preferably at least about 5 hours and most preferably at least about 6 hours.

According to another embodiment of the present invention, there is provided a delayed onset modified release formulation for providing an increased blood concentration of a statin and/or active forms of the statin, relative to that resulting from the administration of an equivalent dose of the conventional immediate release formulations, comprising: a swellable, rapidly disintegrating core comprising at least one statin and at least one release controlling agent and an outer coating over the core, providing delayed release.

According to yet another embodiment of the present invention, such a delayed onset modified release formulation features an erodible film outer coating over the core, providing delayed release. Optionally the outer coating features a pH dependent film coating. Also optionally and alternatively the outer coating features a combination of a water soluble polymer and/or a water swellable hydrophilic polymer and a water insoluble polymer.

According to yet another embodiment of the present invention, there is provided a formulation featuring a burst release core with a coating selected from the group consisting of a pH dependent coating film, preferably an enteric coating; a combination of at least one water soluble polymer and at least one water insoluble polymer; a combination of at least one swellable polymer and at least one water insoluble polymer; a combination of at least a water soluble pore forming agent and at least one water insoluble polymer; at least one swellable gel forming polymer; at least one erodible polymer; a combination of at least one pH dependent polymer and at least one water insoluble polymer; or a two-layer coating comprising a rupturable outer layer and swellable inner layer, wherein the formulation releases substantially no statin in vitro for at least about 1 hour, preferably for at least about 90 minutes and more preferably for at least about 2 hours. Optionally and preferably, at least about 60% of the statin is released in vitro about one hour after the delayed burst release occurs.

According to other embodiments of the present invention, any of the above described formulations may optionally be used for reducing stress on the liver of the subject treated by at least one other drug involved in liver metabolism when administering a statin.

According to yet other embodiments of the present invention, any of the above described formulations may optionally be used for reducing liver side effects including increased level of transaminases when administering a statin.

According to yet other embodiments of the present invention, any of the above described formulations may optionally be used for reducing muscle pain and/or level of CPK when administering a statin.

According to yet other embodiments of the present invention, any of the above described formulations may optionally be used for reducing gastrointestinal effects comprising one or more of nausea, dyspepsia, flatulence or constipation when administering a statin.

According to yet other embodiments of the present invention, any of the above described formulations may optionally be used for providing release of a statin or a pharmaceutically acceptable salt or ester or active form thereof that is not affected by food intake.

According to still other embodiments of the present invention, any of the above described formulations may optionally be characterized in that the in vivo blood plasma concentration of the statin and/or a pharmaceutically acceptable salt and/or ester thereof is substantially zero for at least about one hour after oral administration and is controlled by the lag time, providing an increased blood concentration of a statin and/or active forms of said statin, relative to that resulting from the administration of an equivalent dose of the conventional immediate release formulations. Optionally and preferably, the in vivo blood plasma concentration is extended at least 24 hours.

According to still other embodiments of the present invention, any of the above described formulations may optionally be characterized in that the statin is released in the small intestine and/or lower gastrointestinal tract resulting in increased formation of intestinally active forms of the statin.

According to still other embodiments of the present invention, any of the above described formulations may optionally be characterized in that the statin is released in the small intestine and/or lower gastrointestinal tract resulting in an increased concentration of at least one active forms in the blood. Optionally the formulation comprises a decreased dosage of the statin and/or the pharmaceutically acceptable salt and/or ester thereof. Preferably, the core comprises a dose of statin of no more than about one-half of a dose as compared to a corresponding immediate release formulation, but wherein a level of at least one statin active form after administration of the formulation is at least about a level of the active metabolite after administration of the corresponding immediate release formulation.

### EXAMPLES

The Examples given below are intended only as illustrations of various embodiments of the present invention, and are not intended to be limiting in any way.

Core Preparation Process:

The cores for all Examples were prepared by wet granulation to form fast disintegrating cores. These examples are intended to be illustrative and are not meant to be limiting in any way. First, Povidone K-30 (binder), citric acid (stabilizer/anti-oxidant) and butyl hydroxyanisole (stabilizer) were dissolved in ethanol by using a mechanical stirrer to obtain a clear solution.

Simvastatin as an exemplary active ingredient was mixed with lactose monohydrate 100M (filler), microcrystalline cellulose PH 101 (burst controlling agent), ascorbic acid (stabilizer/anti-oxidant) and croscarmellose sodium (as disintegrant), the mixture was granulated through wet granulation by adding the granulation solution into the granulator. The granulate was dried over a fluidized bed granulator. The dried granulation blend was milled to obtain the desired particle size distribution of the final granulation blend.

Next, the process of blending was performed for the second part of the core. Colloidal silicon dioxide (flow regulating agent) was mixed with an additional amount of croscarmellose sodium (disintegrant) and sieved by a mechanical sieve equipped with a 850 micron screen into the previously obtained granulation blend. The obtained mixture was blended and microcrystalline cellulose (burst controlling agent; Avicel was used) was added into the mixture.

Magnesium stearate, which serves as lubricant, was passed through a mechanical sieve equipped with a 600 micron screen into the mixture and blended for 5 min. This last process formed the tabletting mixture.

The tabletting mixture was then compressed with a Kilian tabletting press equipped with a suitable punches set, such that the average weight of tablet would include a proper amount of the active material, with a hardness sufficient for subsequent coating.

TABLE 1
The formulation of fast disintegrating core used for all Examples.

| Excipient | mg/tab | % |
|---|---|---|
| Core | | % of core |
| Simvastatin | 10.00 | 3.33% |
| Microcrystalline cellulose | 21.00 | 7.00% |
| Lactose monohydrate | 27.00 | 9.00% |
| Butyl Hydroxyanysole (BHA) | 0.12 | 0.04% |
| Citric acid | 3.75 | 1.25% |
| Ascorbic acid | 7.50 | 2.50% |
| Polyvinyl pyrrolidone (Povidone) | 2.20 | 0.73% |
| Croscarmellose sodium | 1.46 | 0.49% |
| Total Granulate | 73.03 | 24.34% |
| | Water + | |
| Granulation solution | ethanol | |
| Croscarmellose sodium | 6.00 | 2.00% |
| Microcrystalline cellulose | 213.20 | 71.06% |
| Microcrystalline cellulose | | |
| Silica colloidal anhyd. | 6.00 | 2.00% |
| Magnesium stearate | 1.80 | 0.60% |
| Total core | 300.0 | 100.00% |
| TCDS Coating | (mg/tab) | % of Coat |
| Microcrystalline cellulose | 19.6 | 57.69% |
| Ethyl Cellulose | 13.1 | 38.46% |
| Cetyl alcohol | 1.3 | 3.85% |
| Total coated tablet | 334.0 | |
| Avicel/EC Rate (Ethylcellulose)/ | 34 | 60/40 |
| Coating weight (mg) | | |

Coating

The formed cores were then coated with different types of coating which cause the formulation to be a delayed burst release formulation, for delayed burst release of the active ingredient. These examples are intended to be illustrative and are not meant to be limiting in any way. The examples of different coatings were prepared as follows.

### Examples 1—A, B and D

### Coating with Kollidon VA 64/Ethyl Cellulose

This coating provides the combination of a water insoluble and a water soluble polymer. Ethyl cellulose (non-swellable water insoluble polymer) was dissolved in ethanol to obtain a clear solution, to which a weighed quantity of Kollidon-VA (a copolymer of polyvinyl pyrrolidone and vinyl acetate) was added and mixed with the mechanical stirrer to complete dissolution. Sieved Talc (glidant or anti adherence) was added and stirred to obtain a homogeneous suspension, which was stirred during the whole coating process.

The coating was performed in a perforated pan coater, with an applied spraying pressure of 0.4 Bar at temperature about 33° C. The coated tablets were dried in an oven at 50° C. for about 16 hours.

The coating formulations are shown in Table 2.

TABLE 2
Different coating formulations used for Example 1

| | A | | B | | D | |
|---|---|---|---|---|---|---|
| | % of | mg/ | % of | mg/ | % of | mg/ |
| Materials | coating | tab | coating | tab | coating | tab |
| Kollidon VA 64 | 16.7% | 12 | 11.1% | 9.3 | 20.0% | 7.4 |
| Ethyl Cellulose 20 | 16.7% | 12 | 22.2% | 18.7 | 40.0% | 14.8 |
| Talc | 66.7% | 48 | 66.7% | 56.0 | 40.0% | 14.8 |
| Total | 100.0% | 72 | 100.0% | 84 | 100.0% | 37 |

### Example 2

### Coating with Hydroxypropyl Methyl Cellulose /Ethyl Cellulose

This coating example provides a combination of at least one swellable polymer and at least one water insoluble polymer. Hydroxypropyl methyl cellulose (HPMC; swellable water soluble polymer) was dissolved in water to obtain a clear solution, to which an aqueous dispersion of Ethyl cellulose with Sodium lauryl sulphate (surfactant) and cetyl alcohol (stiffening agent) was added and mixed with the mechanical stirrer for 30 minutes. Sieved Talc (glidant) was added and stirred to obtain a homogeneous suspension, which was stirred during the whole coating process.

The coating was performed in a perforated pan coater, with an applied spraying pressure of 1.5-2 Bar at temperature about 40° C. The coated tablets were dried in oven at 60° C. for about 16 hours. The coating formulation is as follows:

TABLE 3
The coating formulation used for Example 2

| Materials | % of coating | mg/tab |
|---|---|---|
| Water | | |
| Hydroxypropyl Methyl cellulose | 23.3% | 11 |
| Ethyl Cellulose 20 | 46.5% | 21 |
| Sodium Lauryl Sulphate | 2.3% | 1 |
| Cetyl alcohol | 4.7% | 2 |
| Talc | 23.3% | 11 |
| Total | 100.0% | 46 |

Dissolution Experiments

The in vitro release of simvastatin from the above-referenced formulations was determined as follows. Each of six simvastatin tablets was inserted into individual dissolution cell each of which contains (for examples 1 and 2) 900 ml buffer USP pH 7.0 with 0.5% Sodium Lauryl Sulphate (SLS). For example 3, the medium was 900 ml buffer USP pH 7 with 0.5% SLS throughout the dissolution test. The sample was stirred with a VanKel basket stirrer (Van Kel Inc., USA). Samples were automatically drawn from each dissolution cell to test tubes at various time points. Samples were analyzed by a UV (ultraviolet) light detection (238 nm) and analysis device (HPLC). The amount of drug released was calculated according to a standard set of calculations that are known in the art.

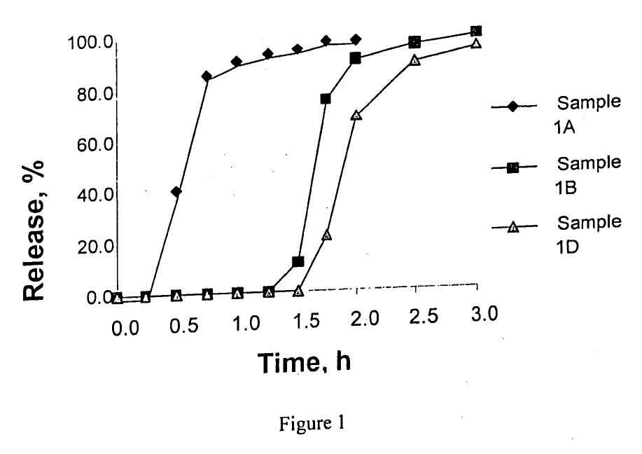

FIG. 1 shows the in vitro dissolution profile for the different coating formulations as described above. The results of release profile are the mean of six tablets for each coating formulation. As can be seen, the burst release occurs after different lag times depending on the coating formulation. Generally, the higher the content of the PVP-VA in the coating composition, the shorter the lag time may be achieved.

TABLE 4
The mean accumulative simvastatin release (%) from
cores coated with different coating formulations
Simvastatin release (%) from tablets coated with Kollidone VA/Ethocel

| Hours | Sample 1A | Sample 1B | Sample 1D |
|---|---|---|---|
| 0.00 | 0.0 | 0.0 | 0.0 |
| 0.25 | 0.0 | 0.0 | 0.0 |
| 0.50 | 40.4 | 0.0 | 0.0 |
| 0.75 | 85.3 | 0.0 | 0.0 |
| 1.00 | 90.6 | 0.0 | 0.0 |
| 1.25 | 93.4 | 0.0 | 0.0 |
| 1.50 | 94.9 | 11.3 | 0.0 |
| 1.75 | 97.2 | 74.8 | 21.4 |
| 2.00 | 97.3 | 89.8 | 68.3 |
| 2.50 | NP | 95.5 | 88.5 |
| 3.00 | NP | 98.9 | 94.2 |

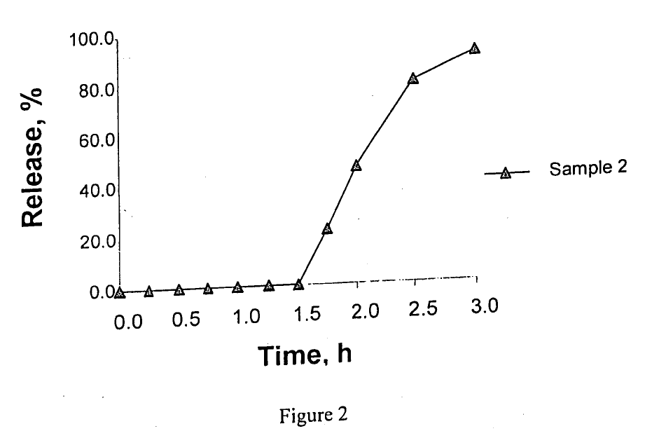

FIG. 2, shows the in vitro dissolution of the coating formulation according to Example 2. As was also seen for the formulation of Example 1, a burst release occurs after a lag time.

TABLE 5
Mean accumulative simvastatin release (%)
from tablets coated with HPMC/Ethocel

| Hours | Sample 2 |
|---|---|
| 0.00 | 0.0 |
| 0.25 | 0.0 |
| 0.50 | 0.0 |
| 0.75 | 0.0 |
| 1.00 | 0.0 |
| 1.25 | 0.0 |
| 1.50 | 0.0 |
| 1.75 | 21.4 |
| 2.00 | 46.2 |
| 2.50 | 79.1 |
| 3.00 | 90.3 |

### Example 3

### Enteric Coating

Triethyl citrate (plasticizer) was dissolved in water to obtain a clear solution, then sodium lauryl sulphate (surfactant) was dissolved in the obtained solution with slow stirring. Hydroxypropylmethyl cellulose acetate succinate (HPMC AS) as a non-limiting example of a pH-dependent coating was added to form an aqueous dispersion. Sieved Talc was added and stirred to obtain a homogeneous suspension, which was stirred during the whole coating process.

The coating was performed in a perforated pan coater, with an applied spraying pressure of 1.5-2 Bar at temperature about 40° C. The coated tablets were dried in oven at 60° C. for about 16 hours. The coating formulation is shown in Table 6:

TABLE 6
Enteric coating example

| Coating | % of coating | mg/tab |
|---|---|---|
| Water | | |
| Hydroxypropylmethyl cellulose Acetate | 55.2% | 40.8 |
| Succinate | | |
| Triethyl Citrate | 15.5% | 11.5 |
| Sodium Lauryl Sulphate | 1.7% | 1.3 |
| Talc | 27.6% | 20.4 |
| Total | 100.0% | 74.0 |

Dissolution tests were performed in apparatus type 1 (baskets), Speed 100 rpm, Medium: 900 ml 0.1 N HCl for 1 hour, then transferred to buffer USP pH 7.0 with 0.5% SLS. The results were analyzed by using the HPLC method. Table 7 shows the dissolution results for enterically coated tablets.

TABLE 7
Dissolution results for enterically coated tablets
Simvastatin release (%) from tablets coated with HPMC AS Medium:
1 hour 0.1 N HCl, then USP buffer phosphate pH 7 with 0.5% SLS

| hours | Sample 3 |
|---|---|
| 0.00 | 0.0 |
| 0.25 | 0.0 |
| 0.50 | 0.0 |
| 0.75 | 0.0 |
| 1.00 | 0.0 |
| 1.25 | 0.0 |
| 1.50 | 51.0 |
| 1.75 | 84.3 |
| 2.00 | 90.3 |
| 2.50 | 96.4 |
| 3.00 | 98.8 |

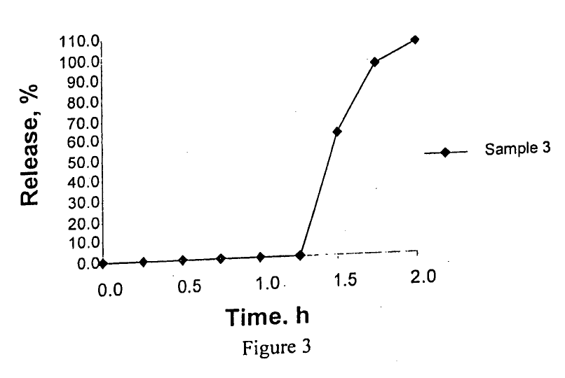

FIG. 3 shows the results of dissolution for a formulation according to the present invention with enterically coated cores. As shown, there is a lag time of approximately 1.25 hours, followed by a rapid release of material.

### Example 4

### Inner Swelling Layer and Outer Water Permeable Layer

This Example provides an optional but preferred embodiment of the present invention, featuring simvastatin tablets with a non-swelling core coated with an inner swelling layer and an outer insoluble water permeable (delay controlling) layer.

TABLE 8
Core composition

| | Weight | |
|---|---|---|
| Product Name | (mg/tab) | % core |
| Simvastatin | 10.00 | 10.0% |
| Microcrystalline cellulose PH 101 | 5.00 | 5.0% |
| Lactose monohydrate 100M | 70.48 | 70.5% |
| Starch 1500 | 10.00 | 10.0% |
| BHA | 0.02 | 0.02% |
| Citric acid | 1.25 | 1.25% |
| Ascorbic acid | 2.50 | 2.50% |
| Magnesium stearate | 0.75 | 0.8% |
| Total Core | 100.0 | 100.0% |

The core in this example was prepared as a granulate comprising Simvastatin, Lactose monohydrate and Microcrystalline cellulose PH 101 (as fillers); Ascorbic acid, Citric acid, and Buthylhydroxyanisole (BHA) as stabilizers (the first two are also anti-oxidants); Pregelatinized Starch as disintegrant and Magnesium stearate as tablet lubricant (added after the wet stage of wet granulation).

The cores were prepared by a wet granulation process. The granulate was dried over a fluidized bed granulator and milled through 813 micron sieve. Magnesium stearate was passed through a mechanical sieve equipped with a 600 micron screen into the granulate and blended for 5 min. The tabletting mixture was then compressed with a Kilian tabletting press equipped with a suitable punches set, such that the average weight of tablet would include a proper amount of the active material, with a hardness sufficient for subsequent coating.

TABLE 9
The inner (swelling) coating:

| Coating | % of coating | mg/tab |
|---|---|---|
| Isopropanol | | |
| Croscarmellose Sodium | 75.0% | 30 |
| Povidon K 30 | 25.0% | 10 |
| Total | 100.0% | 40 |

Povidon K 30 (polyvinylpyrollidone) was dissolved in isopropanol to obtain a clear solution, to which Sodium Croscarmellose was added and mixed with the mechanical stirrer for 30 minutes to form a homogenous suspension, which was stirred during the whole coating process.

TABLE 10
The outer (delay controlling) coating:

| Coating | % of coating | mg/tab |
|---|---|---|
| Water | | |
| Hydroxypropyl Methyl cellulose | 30.3% | 9 |
| Ethyl Cellulose 20 | 60.6% | 18 |
| Sodium Lauryl Sulphate | 3.0% | 1 |
| Cetyl alcohol | 6.1% | 2 |
| Total | 100.0% | 30 |

Hydroxypropyl methyl cellulose (HPMC) was dissolved in water to obtain a clear solution, to which an aqueous dispersion of Ethyl cellulose with Sodium lauryl sulphate and Cetyl alcohol was added and mixed with the mechanical stirrer for 30 minutes to obtain a homogeneous suspension, which was stirred during the whole coating process.

The coatings were performed in a perforated pan coater, with an applied spraying pressure of 1-1.5 Bar at temperature about 40° C. The coated tablets were dried in oven at 60° C. for about 16 hours.

Dissolution tests were performed in apparatus type 1 (baskets), Speed 100 rpm, Medium: 900 ml buffer USP pH 7.0 with 0.5% SLS. The results were analyzed using the HPLC method.

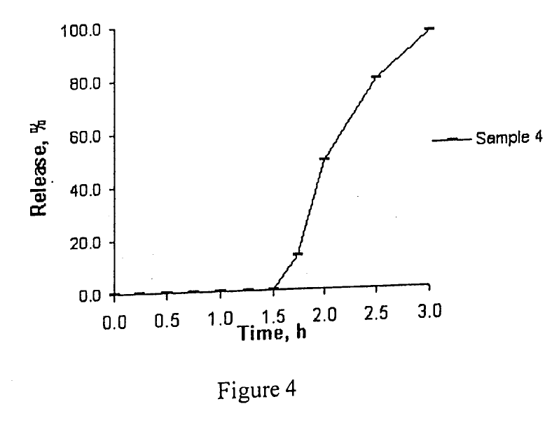

FIG. 4 shows Simvastatin release (%) from tablets coated with inner swelling layer and outer water permeable layer. FIG. 4 shows that there is a lag time of about 1.5 hours for tables prepared according to this Example, followed by a rapid release of material.

TABLE 11
Simvastatin release (%) from tablets coated with inner
swelling layer and outer water permeable layer

| hours | Sample 4 |
|---|---|
| 0.00 | 0.0 |
| 0.25 | 0.0 |
| 0.50 | 0.0 |
| 0.75 | 0.0 |
| 1.00 | 0.0 |
| 1.25 | 0.0 |
| 1.50 | 0.0 |
| 1.75 | 13.0 |
| 2.00 | 48.3 |
| 2.50 | 78.6 |
| 3.00 | 95.8 |

### Bioavailability Study

A randomized, pharmacokinetic pilot study is undertaken to evaluate the bioavailability of test formulations of HMG-CoA reductase inhibitors (statins), and also to determine the levels of the main metabolite, if relevant. For example the study is performed on simvastatin and its main metabolite simvastatin hydroxy acid. For the first study, 40 mg simvastatin tablets are prepared according to Example 3, and for the second study two batches of 16 mg tablets are prepared.

Efficacy Study

The formulation of the present invention is believed to have increased efficacy and to be capable of providing at least similar, if not greater, pharmaceutical effects with the active ingredient with a significantly decreased dosage amount as compared to other orally administered formulations that are known in the art. Without wishing to be limited by a single hypothesis, it is also possible that lower side effects may be observed with the formulation of the present invention, again as compared to other orally administered dosage forms that are known in the art.

Optionally, such a decreased dosage amount of the active ingredient, preferably simvastatin, comprises up to about 60% of the regular dosage amount, more preferably up to about 50% and most preferably up to about 40% of the regular dosage amount. Optionally, the dosage amount comprises up to about 30% of the regular dosage amount. One non-limiting example of a “regular” dosage amount is that administered with the currently available reference product, which as noted above is the Zocor (immediate release) product of Merck. Any other immediate release product could also be considered to be a “regular” product that is known in the art. The dosage amount during a 24 hour period is also determined by the dosage frequency; preferably, the formulation of the present invention is not administered more frequently than the “regular” orally administered formulations; more preferably, the formulation of the present invention is administered once daily, optionally in the evening.

A clinical study studies the issue of both pharmaceutical efficacy as well as bioavailability. This study compares the efficacy and pharmacokinetic parameters of a tablet according to the present invention, with the Zocor reference product (also as used in the bioavailability studies above) which contains a regular dosage of simvastatin. The clinical study is conducted with patients suffering from hypercholesterolemia, although it should be noted that this is for the purpose of the study only and is not intended to be limiting in any way.

The primary end point criteria of the study is equivalent or superior mean percent reductions from baseline (i.e. before the study) in LDL-C (LDL (low density lipoprotein) concentrations in the blood) observed in patients taking the tablet according to the present invention, as compared to the reference product.

Both sets of patients take one tablet per day (present invention or reference) in the evening. Each set includes 80 patients having elevated cholesterol levels. The patients either have not been previously treated with a statin, or are undergoing a 6 week washout period (during which no statin is given) before the study begins. The study is double-blind, randomized and multicenter. Any potential adverse effects are detected with clinical and laboratory testing.

A treatment period of 6 weeks occurs, with periodic measurements of the blood.

As noted above, the clinical study shows that the tablet of the present invention (with the lower dosage amount of 10 to 12 mg per tablet) is at least as pharmaceutically effective as the immediate release reference product (with 20 mg per tablet), thereby providing at least similar clinical efficacy but with a significantly lower dose (up to about 40% of the immediate release reference product).

Treatment with the tablet of the present invention has at least similar effects as in published literature/studies, although the final comparison is made with the set of patients who are taking the reference product, Zocor®, within the study itself.

The formulation of the present invention therefore provides a delayed onset, modified release formulation for delivery of statins in the lower GI tract preferentially to the colon or small intestine, which provides higher blood levels of statin or its metabolites in the bloodstream in comparison to a conventional immediate release formulation. The bioavailability is shown to be higher than that of a known reference product. The formulations according to the present invention should result in fewer side effects, greater safety, efficacy, and patient compliance.

The formulation of the present invention preferably comprises a delayed onset, modified release formulation, which is not a delayed burst release or delayed immediate or fast release formulation. The release is designed to occur within a period of less than 8 hours following oral administration, preferably with selective absorption of the active agent in the lower GI tract.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A delayed burst release formulation for providing an increased blood concentration of a statin and/or active forms of said statin, relative to that resulting from the administration of an equivalent dose of the conventional immediate release formulations, comprising:
a core and an outer coating that surrounds the core;
said core comprising a statin and/or a pharmaceutically acceptable salt and/or ester thereof, said core comprising a burst controlling agent and a disintegrant, and
said coating characterized by at least one of the following:
a. pH dependent coating film, preferably an enteric coating;
b. a combination of at least one water soluble polymer and at least one water insoluble polymer;
c. a combination of at least one swellable polymer and at least one water insoluble polymer;
d. a combination of at least a water soluble pore forming agent and at least one water insoluble polymer;
e. at least one swellable gel forming polymer;
f. at least one biodegradable polymer;
g. at least one erodible polymer;
h. a combination of at least one pH dependent polymer and at least one water insoluble polymer;
i. a two-layer coating comprising a rupturing outer layer and swellable inner layer.

2. The formulation of claim 1, wherein said formulation preferentially releases statin in the intestine of the subject.

3. The formulation of claim 1, wherein said formulation preferentially releases statin in the lower gastrointestinal tract.

4. The formulation of claim 1, wherein said formulation preferentially releases statin in the colon of the subject.

5. The formulation of claim 1 wherein said core is in the form of one of a tablet, pellets, microparticles, agglomerate, and capsule.

6. The formulation of claim 1, wherein said burst controlling agent comprises a water insoluble polymer.

7. The formulation of claim 6, wherein said water insoluble polymer is selected from the group consisting of cross-linked polysaccharide, water insoluble starch, microcrystalline cellulose, water insoluble cross-linked peptide, water insoluble cross-linked protein, water insoluble cross-linked gelatin, water insoluble cross-linked hydrolyzed gelatin, water insoluble cross-linked collagen, modified cellulose, and cross-linked polyacrylic acid.

8. The formulation of claim 7 wherein said cross-linked polysaccharide is selected from the group consisting of insoluble metal salts or cross-linked derivatives of alginate, pectin, xantham gum, guar gum, tragacanth gum, and locust bean gum, carrageenan, metal salts thereof, and covalently cross-linked derivatives thereof.

9. The formulation of claim 7 wherein said modified cellulose is selected from the group consisting of cross-linked derivatives of hydroxypropylcellulose, hydroxypropylmethylcellulose, hydroxyethylcellulose, methylcellulose, carboxymethylcellulose, and metal salts of carboxymethylcellulose.

10. The formulation of claim 6, wherein said water insoluble polymer is calcium pectinate.

11. The formulation of claim 6, wherein said water insoluble polymer is microcrystalline cellulose.

12. The formulation of claim 1, wherein said water insoluble polymer is swellable.

13. The formulation of claim 1, wherein said water insoluble polymer is non swellable.

14. The formulation of claim 1, wherein said water insoluble polymer is hydrophobic.

15. The formulation of claim 1, wherein said disintegrant is selected from the group consisting of cross-linked polyvinylpyrrolidinone, sodium starch glycolate, cross-linked sodium carboxymethylcellulose, pregelatinized starch, microcrystalline starch, water insoluble starch, calcium carboxymethylcellulose, magnesium aluminium silicate, and combinations thereof.

16. The formulation of claim 1, wherein said core further comprises at least one of an absorption enhancer, a binder, a hardness enhancing agent and an excipient.

17. The formulation of claim 16, wherein said binder is selected from the group consisting of starch, polyvinylpyrrolidone, low molecular weight hydroxypropylcellulose, low molecular weight hydroxypropylmethylcellulose, low molecular weight carboxymethylcellulose, ethylcellulose, gelatin, polyethylene oxide, acacia, dextrin, magnesium aluminum silicate, and polymethacrylates.

18. The formulation of claim 16, wherein said hardness enhancing agent is microcrystalline cellulose.

19. The formulation of claim 1, wherein said core further comprises a buffering agent.

20. The formulation of claim 19, wherein said buffering agent is selected from the group consisting of an inorganic salt compound and an organic alkaline salt compound.

21. The formulation of claim 1, wherein said core further comprises a filler.

22. The formulation of claim 21, wherein said filler is selected from the group consisting of, starch, lactitol, lactose, an inorganic calcium salt, sucrose, and combinations thereof.

23. The formulation of claim 1, wherein said core further comprises a flow regulating agent.

24. The formulation of claim 23, wherein said flow regulating agent includes at least one of colloidal silicon dioxide and aluminum silicate.

25. The formulation of claim 1, wherein said core further comprises a lubricant.

26. The formulation of claim 25, wherein said lubricant is selected from the group consisting of stearate salts; stearic acid, talc, sodium stearyl fumarate, sodium lauryl sulfate, polyethylene glycol, and glycerol behenate, or a combination thereof.

27. The formulation of claim 1, wherein said coating comprises a pH dependent polymer.

28. The formulation of claim 27, wherein said pH dependent polymer is selected from the group consisting of methacrylic acid copolymers, ammonio methacrylate co-polymers, or a mixture thereof.

29. The formulation of claim 27, wherein said pH dependent polymer is selected from the group consisting of a hydroxypropylmethyl cellulose phthalate, polyvinyl acetate phthalate, cellulose acetate phthalate, hydroxypropylmethyl cellulose acetate succinate, poly(methacrylic acid, methyl methacrylate)1:1 and poly(methacrylic acid, ethyl acrylate)1:1, alginic acid, and sodium alginate.

30. The formulation of claim 27, wherein said pH dependent coating comprises an enteric coating.

31. The formulation of claim 30, wherein said enteric coating comprises Hydroxypropylmethyl cellulose acetate succinate (HPMC AS).

32. The formulation of claim 31, wherein said HPMC AS is present in an amount ranging from about 25% to about 90% of said enteric coating.

33. The formulation of any of claims 30-32, wherein said enteric coating further comprises a plasticizer.

34. The formulation of claim 33, wherein said plasticizer comprises triethyl citrate.

35. The formulation of any of claims 30-34, wherein said enteric coating further comprises a surfactant.

36. The formulation of claim 35, wherein said surfactant comprises sodium lauryl sulfate.

37. The formulation of claim 1, wherein said coating comprises a combination of at least one water soluble polymer and at least one water insoluble polymer.

38. The formulation of claim 37, wherein said water-soluble polymer is selected from the group consisting of polyvinyl alcohol, polyvinylpyrrolidone (PVP), methylcellulose, hydroxypropylcellulose, hydroxypropylmethyl cellulose, polyethylene glycol, carboxymethyl cellulose (sodium salt), hydroxyethyl cellulose, a water soluble gum, polysaccharide and/or mixtures thereof.

39. The formulation of claim 37, wherein said water insoluble polymer is selected from the group consisting of a podimethylaminoethylacrylate/ethylmethacrylate copolymer, an ethylmethacrylate/chlorotrimethylammoniumethyl methacrylate copolymer, a dimethylaminoethylmethacrylate/methylmethacrylate and butylmethacrylate copolymer, a copolymer based on neutral methacrylic acid esters and dimethylaminoethyl methacrylate esters, an ethylacrylate and methylacrylate/ethylmethacrylate and methyl methylacrylate copolymer, ethylcellulose, shellac, zein, and waxes, paraffin, cellulose acetate, cellulose propionate, cellulose acetate propionate, cellulose acetate butyrate, cellulose acetate phthalate, cellulose triacetate, poly(methyl methacrylate), poly(ethylmethacrylate), poly(butyl methacrylate), poly(isobutyl methacrylate), and poly(hexyl methacrylate), poly(isodecyl methacrylate), poly(lauryl methacrylate), poly(phenyl methacrylate), poly (methylacrylate), poly(isopropyl acrylate), poly(isobutyl acrylate) poly(octadecyl acrylate), poly(ethylene), poly(ethylene) low density, poly(ethylene) high density, poly (ethylene oxide), poly(ethyleneterephthalate), poly(vinyl isobutyl ether), poly(vinyl acetate), poly(vinyl chloride) and polyurethane, and/or mixtures thereof.

40. The formulation of claim 39, wherein said water insoluble polymer comprises ethylcellulose.

41. The formulation of claim 38, wherein said water soluble polymer comprises a copolymer of polyvinyl pyrrolidone and vinyl acetate.

42. The formulation of claim 37, wherein said water insoluble polymer is present in an amount ranging from about 20% to about 95% of said coating, and said water soluble polymer is present in an amount ranging from about 5% to about 45% of said coating.

43. The formulation of any of claims 30-32, wherein said coating further comprises a glidant.

44. The formulation of claim 43, wherein said glidant comprises sieved talc.

45. The formulation of claim 1, wherein said coating comprises a combination of at least a water soluble pore forming agent and at least one water insoluble polymer.

46. The formulation of claim 45, wherein said pore-forming agent is selected from the group consisting of saccharose, sodium chloride, potassium chloride, polyvinylpyrrolidone, and/or polyethyleneglycol, water soluble organic acids, sugars and sugar alcohol.

47. The formulation of claim 45, wherein said pore forming compound is distributed uniformly throughout said water insoluble polymer.

48. The formulation of claim 45, wherein said pore forming compound is distributed randomly throughout said water insoluble polymer.

49. The formulation of claim 45, wherein said pore-forming compound comprises about 1 part to about 35 parts for each about 1 to about 10 parts of said water insoluble polymer.

50. The formulation of claim 1, wherein said coating comprises an erodible polymer.

51. The formulation of claim 50, wherein said erodible composition comprises at least one of a slow dissolving and a slow disintegrating composition.

52. The formulation of claim 50, wherein said erodible composition comprises at least one of a slowly water soluble polymer and a swellable polymer

53. The formulation of claims 51 or 52, wherein said erodible composition further comprises a disintegrant.

54. The formulation of claim 1, wherein said coating comprises at least one swellable gel forming polymer.

55. The formulation of claim 54, wherein said swellable gel-forming polymer is selected from the group consisting of cellulosic polymers; vinyl polymers; acrylic polymers and copolymers, methacrylic acid copolymers, ethyl acrylate-methyl methacrylate copolymers, natural and synthetic gums, gelatin, collagen, proteins, polysaccharides, pectin, pectic acid, alginic acid, sodium alginate, carbopol, polyaminoacids, polyalcohols, polyglycols; and mixtures thereof.

56. The formulation of claim 55, wherein said cellulosic polymer is selected from the group consisting of methylcellulose, carboxymethylcellulose, hydroxypropylcellulose, hydroxypropylmethylcellulose, and hydroxyethylcellulose.

57. The formulation of claim 56, wherein said cellulosic polymer comprises hydroxymethylcellulose.

58. The formulation of claim 55, wherein said swellable gel-forming polymer comprises carbopol.

59. The formulation of claim 1, wherein said coating further comprises at least one of a lubricant, a flow promoting agent, a plasticizer, an antisticking agent, natural and synthetic flavorings and natural and synthetic colorants.

60. The formulation of claim 59, wherein said lubricant further comprises at least one of polyethylene glycol, polyvinylpyrrolidone, talc, magnesium stearate, glyceryl behenate, stearic acid, and titanium dioxide.

61. The formulation of claim 1, wherein said coating comprises a combination of at least one swellable polymer and at least one water insoluble polymer.

62. The formulation of claim 61, wherein said swellable polymer comprises hydroxypropyl methyl cellulose (HPMC).

63. The formulation of claim 62, wherein said water insoluble polymer comprises ethyl cellulose.

64. The formulation of claim 63, wherein said water insoluble polymer is present in an amount ranging from about 20% to about 95%, and said swellable polymer is present in an amount ranging from about 5% to about 45% of the coating.

65. The formulation of any of claims 61-64, wherein the coating further comprises a surfactant.

66. The formulation of claim 65, wherein said surfactant comprises sodium lauryl sulphate.

67. The formulation of any of claims 61-66, wherein the coating further comprises a stiffening agent.

68. The formulation of claim 67, wherein said stiffening agent comprises cetyl alcohol.

69. The formulation of any of claims 61-68, wherein the coating further comprises a glidant.

70. The formulation of claim 69, wherein said glidant comprises sieved talc.

71. The formulation of claim 1, wherein said coating comprises a combination of at least one pH dependent polymer and at least one water insoluble polymer.

72. The formulation of claim 1, wherein said coating comprises a two-layer coating comprising a rupturable outer layer and swellable inner layer.

73. The formulation of claim 72, wherein said two-layer coating ruptures independently of said core.

74. The formulation of any one of claims 72 or 73, wherein said inner layer comprises a disintegrant.

75. The formulation of any of claims 72-74, wherein said inner layer comprises at least one polymer being able to swell when contacted by water.

76. The formulation of claim 75, wherein said at least one polymer is selected from the group consisting of hydroxypropylmethyl cellulose, high molecular weight of carboxymethyl cellulose, high molecular weight of hydroxypropyl cellulose, high molecular weight of hydroxyethyl cellulose, high molecular weight of hydroxymethyl cellulose, polyhydroxyethyl methacrylate, polyhydroxymethyl methacrylate, polyacrylic acid, carbopole, polycarbophil, gums, polysaccharides, modified polysaccharides, cross-linked polysaccharide, water insoluble starch, microcrystalline cellulose, water insoluble cross-linked peptide, water insoluble cross-linked protein, water insoluble cross-linked gelatin, water insoluble cross-linked hydrolyzed gelatin, water insoluble cross-linked collagen modified cellulose, and cross-linked polyacrylic acid.

77. The formulation of claim 76, wherein said cross-linked polysaccharide is selected from the group consisting of insoluble metal salts or cross-linked derivatives of alginate, pectin, xanthan gum, guar gum, tragacanth gum, and locust bean gum, carrageenan, metal salts thereof, and covalently cross-linked derivatives thereof.

78. The formulation of claim 76, wherein said modified cellulose is selected from the group consisting of cross-linked derivatives of hydroxypropylcellulose, hydroxypropylmethylcellulose, hydroxyethylcellulose, methylcellulose, carboxymethylcellulose, and metal salts of carboxymethylcellulose.

79. The formulation of any of claims 74-78, wherein said inner layer comprises a disintegrant embedded in a water soluble film forming polymer.

80. The formulation of any of claims 74-78, wherein said inner layer comprises a combination of a water soluble polymer forming a film matrix, and a swellable water insoluble polymer particulate embedded into said film matrix.

81. The formulation of any of claims 72-80, wherein said rupturable outer layer comprises a brittle polymer.

82. The formulation of any of claims 72-81, wherein said rupturable outer layer comprises at least one permeation-enhancing agent.

83. The formulation of claims 81 or 82, wherein said rupturable outer layer comprises a water insoluble polymer selected from the group consisting of a dimethylaminoethylacrylate/ethylmethacrylate copolymer, the copolymer being based on acrylic and methacrylic acid esters with a low content of quaternary ammonium groups, wherein the molar ratio of the ammonium groups to the remaining neutral (meth)acrylic acid esters is approximately 1:20, the polymer corresponding to USP/NF “Ammonio Methacrylate Copolymer Type A”, an ethylmethacrylate/chlorotrimethylammoniumethyl methacrylate copolymer, the copolymer based on acrylic and methacrylic acid esters with a low content of quaternary ammonium groups wherein the molar ratio of the ammonium groups to the remaining neutral (meth)acrylic acid esters is 1:40, the polymer corresponding to USP/NF “Ammonio Methacrylate Copolymer Type B”, a dimethylaminoethylmethacrylate/methylmethacrylate and butylmethacrylate copolymer, a copolymer based on neutral methacrylic acid esters and dimethylaminoethyl methacrylate esters wherein the polymer is cationic in the presence of acids, an ethylacrylate and methylacrylate/ethylmethacrylate and methyl methylacrylate copolymer, the copolymer being a neutral copolymer based on neutral methacrylic acid and acrylic acid esters, ethylcellulose, shellac, zein, and waxes.

84. The formulation of claim 83, wherein said water insoluble polymer comprises ethylcellulose.

85. The formulation of claim 1, wherein said coating is a compression coating.

86. The formulation of claim 85, wherein said coating comprises a gum selected from the group consisting of xanthan gum, locust bean gum, galactans, mannans, alginates, gum karaya, pectin, agar, tragacanth, accacia, carrageenan, tragacanth, chitosan, agar, alginic acid, hydrocolloids acacia catechu, salai guggal, indian bodellum, copaiba gum, asafetida, cambi gum, Enterolobium cyclocarpum, mastic gum, benzoin gum, sandarac, gambier gum, butea frondosa (Flame of Forest Gum), myrrh, konjak mannan, guar gum, welan gum, gellan gum, tara gum, locust bean gum, carageenan gum, glucomannan, galactan gum, sodium alginate, tragacanth, chitosan, xanthan gum, deacetylated xanthan gum, pectin, sodium polypectate, gluten, karaya gum, tamarind gum, ghatti gum, Accaroid/Yacca/Red gum, dammar gum, juniper gum, ester gum, ipil-ipil seed gum, gum talha (acacia seyal), and cultured plant cell gums including those of the plants of the genera: acacia, actinidia, aptenia, carbobrotus, chickorium, cucumis, glycine, hibiscus, hordeum, letuca, lycopersicon, malus, medicago, mesembryanthemum, oryza, panicum, phalaris, phleum, poliathus, polycarbophil, sida, solanum, trifolium, trigonella, Afzelia africana seed gum, Treculia africana gum, detarium gum, cassia gum, carob gum, Prosopis africana gum, Colocassia esulenta gum, Hakea gibbosa gum, khaya gum, scleroglucan, zea, mixtures of any of the foregoing.

87. The formulation of claim 86, wherein said compression coating comprises at least one of a heteropolysaccharide and a homopolysaccharide, or a mixture thereof.

88. The formulation of claim 87, wherein said heteropolysaccharide is xanthan gum.

89. The formulation of claim 1, wherein said outer coating further comprises a plasticizer.

90. The formulation of claim 89, wherein said plasticizer is selected from the group consisting of dibutyl sebacate, polyethylene glycol and polypropylene glycol, dibutyl phthalate, diethyl phthalate, triethyl citrate, tributyl citrate, acetylated monoglyceride, acetyl tributyl citrate, triacetin, dimethyl phthalate, benzyl benzoate, butyl and/or glycol esters of fatty acids, refined mineral oils, oleic acid, castor oil, corn oil, camphor, glycerol and sorbitol or a combination thereof.

91. The formulation of claim 1, wherein said coating further comprises a stiffening agent.

92. The formulation of claim 91, wherein said stiffening agent comprises cetyl alcohol.

93. The formulation of claim 1, wherein said coating and/or said core further comprises at least one of a wetting agent, a suspending agent, and a dispersing agent, or a combination thereof.

94. The formulation of claim 93 wherein said wetting agent is selected from the group consisting of poloxamer, polyoxyethylene ethers, polyoxyethylene sorbitan fatty acid esters, polyoxymethylene stearate, sodium lauryl sulfate, sorbitan fatty acid esters, benzalkonium chloride, polyethoxylated castor oil, and docusate sodium.

95. The formulation of claim 93, wherein said suspending agent is selected from the group consisting of alginic acid, bentonite, carbomer, carboxymethylcellulose, carboxymethylcellulose calcium, hydroxyethylcellulose, hydroxypropylcellulose, microcrystalline cellulose, colloidal silicon dioxide, dextrin, gelatin, guar gum, xanthan gum, kaolin, magnesium aluminum silicate, maltitol medium chain triglycerides, methylcellulose, polyoxyethylene sorbitan fatty acid esters, polyvinylpyrrolidinone, propylene glycol alginate, sodium alginate, sorbitan fatty acid esters, and tragacanth.

96. The formulation of claim 95, wherein said dispersing agent is selected from the group consisting of poloxamer, polyoxyethylene sorbitan fatty acid esters and sorbitan fatty acid esters.

97. The formulation of claim 1, further comprising an enteric coating disposed over said coating.

98. The formulation of claim 97, wherein said enteric coating is selected from the group consisting of cellulose acetate phthalate, hydroxy propyl methyl cellulose acetate succinate, poly(methacrylic acid, methyl methacrylate)1:1 and (Eudragit® L100), poly(methacrylic acid, ethyl acrylate) 1:1 (Eudragit® L30D-55).

99. The formulation of claim 1, wherein said core further comprises a synergistic agent (sequestrate).

100. The formulation of claim 99, wherein said synergistic agent is selected from the group consisting of citric acid and ascorbic acid.

101. The formulation of claim 1, wherein said core further comprises an antioxidant.

102. The formulation of claim 101, wherein said antioxidant is selected from the group consisting of 4,4 (2,3 dimethyl tetramethylene dipyrochatechol), Tocopherol-rich extract (natural vitamin E), α-tocopherol (synthetic Vitamin E), β-tocopherol, γ-tocopherol, δ-tocopherol, Butylhydroxinon, Butyl hydroxyanisole (BHA), Butyl hydroxytoluene (BHT), Propyl Gallate, Octyl gallate, Dodecyl Gallate, Tertiary butylhydroquinone (TBHQ), Fumaric acid, Malic acid, Ascorbic acid (Vitamin C), Sodium ascorbate, Calcium ascorbate, Potassium ascorbate, Ascorbyl palmitate, Ascorbyl stearate, Citric acid, Sodium lactate, Potassium lactate, Calcium lactate, Magnesium lactate, Anoxomer, Erythorbic acid, Sodium erythorbate, Erythorbin acid, Sodium erythorbin, Ethoxyquin, Glycine, Gum guaiac, Sodium citrates (monosodium citrate, disodium citrate, trisodium citrate), Potassium citrates (monopotassium citrate, tripotassium citrate), Lecithin, Polyphosphate, Tartaric acid, Sodium tartrates (monosodium tartrate, disodium tartrate), Potassium tartrates (monopotassium tartrate, dipotassium tartrate), Sodium potassium tartrate, Phosphoric acid, Sodium phosphates (monosodium phosphate, disodium phosphate, trisodium phosphate), Potassium phosphates (monopotassium phosphate, dipotassium phosphate, tripotassium phosphate), Calcium disodium ethylene diamine tetra-acetate (Calcium disodium EDTA), Lactic acid, Trihydroxy butyrophenone and Thiodipropionic acid.

103. The formulation of claim 1, wherein said core further comprises a chelating agent.

104. The formulation of claim 103, wherein said chelating agent comprises an anti-oxidant.

105. The formulation of claim 104, wherein said antioxidant is selected from the group consisting of antioxidants, dipotassium edentate, disodium edentate, edetate calcium disodium, edetic acid, fumaric acid, malic acid, maltol, sodium edentate, and trisodium edetate.

106. The formulation of claim 1, wherein the formulation releases substantially no statin in vitro for at least about 1 hour.

107. The formulation of claim 1, wherein the formulation releases substantially no statin in vitro for at least about 90 minutes.

108. The formulation of claim 1, wherein the formulation releases substantially no statin in vitro for at least about 2 hours.

109. The formulation of claim 1, wherein at least about 60% of the statin is released in vitro about one hour after said delayed burst release occurs.

110. The formulation of claim 1, characterized in that the in vivo blood plasma concentration of statin and/or a pharmaceutically acceptable salt and/or ester thereof is substantially zero for at least about two hours after oral administration and is controlled by the lag time, providing an increased blood concentration of a statin and/or active forms of said statin, relative to that resulting from the administration of an equivalent dose of the conventional immediate release formulations.

111. The formulation of claim 110, characterized in that the formulation provides an increased blood concentration of a statin and/or active forms of said statin, relative to that resulting from the administration of an equivalent dose of the conventional immediate release formulations

112. The formulation of claim 111, wherein said in vivo blood plasma concentration is extended at least 24 hours.

113. The formulation of any of the preceding claims wherein said statin is released in the small intestine and/or lower gastrointestinal tract resulting in increased formation of intestinally active forms of said statin.

114. The formulation of any of the preceding claims wherein said statin is released in the small intestine and/or lower gastrointestinal tract resulting in an increased concentration of at least one active forms in the blood.

115. The formulation of claim 114, wherein said formulation comprises a decreased dosage of said statin and/or said pharmaceutically acceptable salt and/or ester thereof.

116. The formulation of claim 114, wherein said core comprises a dose of statin of no more than about one-half of a dose as compared to a corresponding immediate release formulation, but wherein a level of at least one statin active form after administration of said delayed burst release formulation is at least about a level of said active metabolite after administration of said corresponding immediate release formulation.

117. A formulation for providing enhanced bioavailability of a statin and/or a pharmaceutically acceptable salt and/or ester thereof in a subject, comprising:
a delayed burst release formulation for oral administration comprising a core and an outer coating that surrounds the core;
said core comprising a statin and/or a pharmaceutically acceptable salt and/or ester thereof, a burst controlling agent and a disintegrant; and
said coating comprising a water-insoluble hydrophobic carrier and a hydrophilic particulate matter;
characterized in that at least about 70% of the statin is released in vitro about one hour after said delayed burst release occurs.

118. A method for providing fast release of statin and/or a pharmaceutically acceptable salt and/or ester thereof in the lower gastrointestinal tract in a subject, comprising:
administering orally to the subject a delayed burst release formulation comprising a core and an outer coating that surrounds the core;
said core comprising statin and/or a pharmaceutically acceptable salt and/or ester thereof and
said coating characterized by at least one of the
a. pH dependent coating film, preferably an enteric coating;
b. a combination of at least one water soluble polymer and at least one water insoluble polymer;
c. a combination of at least one swellable polymer and at least one water insoluble polymer;
d. a combination of at least a water soluble pore forming agent and at least one water insoluble polymer;
e. at least one swellable gel forming polymer;
f. at least one biodegradable polymer;
g. at least one erodible polymer;
h. a combination of at least one pH dependent polymer and at least one water insoluble polymer;
i. a two-layer coating comprising a rupturable outer layer and swellable inner layer; and
characterized in that the in vivo blood plasma concentration of statin and/or a pharmaceutically acceptable salt and/or ester thereof is substantially zero for at least about two hours after oral administration and is controlled by the lag time, providing an increased blood concentration of a statin and/or active forms of said statin, relative to that resulting from the administration of an equivalent dose of the conventional immediate release formulations.

119. The method of claim 118, wherein said in vivo blood plasma concentration is extended for at least 24 hours.

120. The method of claims 118 or 119, wherein said statin is released in small intestine and/or lower gastrointestinal tract including colon resulting in an increased intestinal metabolite formation.

121. The method of any of claims 118-120 wherein said statin is released in small intestine and/or lower gastrointestinal tract including colon resulting in an increased concentration of a metabolite in the blood.

122. A method for using a modified release formulation according to any of the preceding claims to reduce stress on the liver of the subject treated by at least one other drug metabolized by the liver when administering a statin.

123. A method for using a modified release formulation according to any of the preceding claims to reduce stress on the liver of the subject when administering a statin.

124. A method for using a modified release formulation according to any of the preceding claims to reduce liver side effects including increased level of transaminases when administering a statin.

125. A method for using a modified release formulation according to any of the preceding claims to reduce muscle pain and/or level of CPK when administering a statin.

126. A method for using a modified release formulation according to any of the preceding claims to reduce gastrointestinal effects comprising one or more of nausea, dyspepsia, flatulence or constipation when administering a statin.

127. A method for using a delayed onset modified release formulation according to any of the preceding claims, for providing release of said statin or said pharmaceutically acceptable salt or ester or active form thereof that is not affected by food.

128. A method for using a delayed onset modified release formulation according to any of the preceding claims, for providing treatment for high blood cholesterol to a subject in need thereof.

129. A method for providing a therapeutically effective amount of a statin and/or a pharmaceutically acceptable salt and/or ester and/or active form thereof to a subject, comprising administering orally to the subject a delayed onset modified release formulation according to any of the preceding claims.

130. A method for providing a delayed burst release of a therapeutically effective amount of a statin and/or a pharmaceutically acceptable salt and/or ester thereof to a subject wherein substantially no statin is released in vitro for at least about two hours.

131. A method for providing enhanced bioavailability of statin and/or a pharmaceutically acceptable salts and/or esters thereof and/or its related metabolite in a subject, comprising:
administering orally to the subject a modified release formulation comprising a core and an outer coating that surrounds the core;
said core comprising a statin, or a pharmaceutically acceptable salt thereof and at least one release controlling agent, and
said coating characterized by at least one of
a. pH dependent coating film, preferably an enteric coating;
b. a combination of at least one water soluble polymer and at least one water insoluble polymer;
c. a combination of at least one swellable polymer and at least one water insoluble polymer;
d. a combination of at least a water soluble pore forming agent and at least one water insoluble polymer;
e. at least one swellable gel forming polymer;
f. at least one biodegradable polymer;
g. at least one erodible polymer;
h. a combination of at least one pH dependent polymer and at least one water insoluble polymer;
i. a two-layer coating comprising a rupturable outer layer and swellable inner layer;
characterized in that the in vivo blood plasma concentration of said statin is substantially zero for at least about two hours after oral administration.

132. A modified release formulation that releases a statin and/or a pharmaceutically acceptable salt and/or ester thereof in the lower gastrointestinal tract of a subject, characterized in that the in vivo blood plasma concentration of said statin and/or a pharmaceutically acceptable salt and/or ester thereof is substantially zero for at least about one hour after oral administration and is controlled by the lag time, providing an increased blood concentration of a statin and/or active forms of said statin, relative to that resulting from the administration of an equivalent dose of the conventional immediate release formulations.

133. The method of claim 132, wherein said in vivo blood plasma concentration is extended at least 24 hours.

134. The method of claim 132, wherein said core comprises a dose of statin of no more than about one-half of a dose as compared to a corresponding immediate release formulation, but wherein a level of at least one statin active form after administration of said modified release formulation is at least about a level of said active metabolite after administration of said corresponding immediate release formulation.

135. The method of claim 132, wherein said core comprises a dose of statin of no more than about one-half of a dose in said corresponding immediate release formulation, but wherein a level of at least one statin active metabolite after administration of said modified release formulation is at least about a level of said active metabolite after administration of said corresponding immediate release formulation.

136. The formulation of any of the preceding claims wherein said statin is released in the small intestine and/or lower gastrointestinal tract resulting in an increased formation of intestinally active forms of said statin.

137. A modified release formulation for providing an increased blood concentration of a statin and/or active forms of said statin, relative to that resulting from the administration of an equivalent dose of the conventional immediate release formulations, comprising:
a core and an outer coating that surrounds the core;
said core comprising a statin and/or a pharmaceutically acceptable salt and/or ester thereof, and
said coating characterized by at least one of the following:
a. pH dependent coating film, preferably an enteric coating;
b. a combination of at least one water soluble polymer and at least one water insoluble polymer;
c. a combination of at least one swellable polymer and at least one water insoluble polymer;
d. a combination of at least a water soluble pore forming agent and at least one water insoluble polymer;
e. at least one swellable gel forming polymer;
f. at least one biodegradable polymer;
g. at least one erodible polymer;
h. a combination of at least one pH dependent polymer and at least one water insoluble polymer;
i. a two-layer coating comprising a rupturing outer layer and swellable inner layer.

138. A modified release formulation for providing an increased blood concentration of a statin and/or active forms of said statin, relative to that resulting from the administration of an equivalent dose of the conventional immediate release formulations, comprising:
a core and an outer coating that surrounds the core;
said core comprising a statin and/or a pharmaceutically acceptable salt and/or ester thereof, and
said coating comprising a two-layer coating comprising a rupturing outer layer and swellable inner layer.

139. The formulation of claim 138, wherein said two-layer coating ruptures independently of said core.

140. The formulation of claims 138 or 139, wherein said inner layer comprises a disintegrant.

141. The formulation of any of claims 138-140, wherein said inner layer comprises at least one polymer being able to swell when contacted by water.

142. The formulation of claim 141, wherein said at least one polymer is selected from the group consisting of hydroxypropylmethyl cellulose, high molecular weight of carboxymethyl cellulose, high molecular weight of hydroxypropyl cellulose, high molecular weight of hydroxyethyl cellulose, high molecular weight of hydroxymethyl cellulose, polyhydroxyethyl methacrylate, polyhydroxymethyl methacrylate, polyacrylic acid, carbopol, polycarbophil, gums, polysaccharides, modified polysaccharides, cross-linked polysaccharide, water insoluble starch, microcrystalline cellulose, water insoluble cross-linked peptide, water insoluble cross-linked protein, water insoluble cross-linked gelatin, water insoluble cross-linked hydrolyzed gelatin, water insoluble cross-linked collagen modified cellulose, and cross-linked polyacrylic acid.

143. The formulation of claim 142, wherein said cross-linked polysaccharide is selected from the group consisting of insoluble metal salts or cross-linked derivatives of alginate, pectin, xanthan gum, guar gum, tragacanth gum, and locust bean gum, carrageenan, metal salts thereof, and covalently cross-linked derivatives thereof.

144. The formulation of claim 143, wherein said modified cellulose is selected from the group consisting of cross-linked derivatives of hydroxypropylcellulose, hydroxypropylmethylcellulose, hydroxyethylcellulose, methylcellulose, carboxymethylcellulose, and metal salts of carboxymethylcellulose.

145. The formulation of any of claims 140-144, wherein said inner layer comprises a disintegrant embedded in a water soluble film forming polymer.

146. The formulation of any of claims 140-144, wherein said inner layer comprises a combination of a water soluble polymer forming a film matrix, and a swellable water insoluble polymer particulate embedded into said film matrix.

147. The formulation of any of claims 138-146, wherein said rupturable outer layer comprises a brittle polymer.

148. The formulation of any of claims 138-147, wherein said rupturable outer layer comprises at least one permeation-enhancing agent.

149. The formulation of claims 147 or 148, wherein said rupturable outer layer comprises a water insoluble polymer selected from the group consisting of a dimethylaminoethylacrylate/ethylmethacrylate copolymer, the copolymer being based on acrylic and methacrylic acid esters with a low content of quaternary ammonium groups, wherein the molar ratio of the ammonium groups to the remaining neutral (meth)acrylic acid esters is approximately 1:20, the polymer corresponding to USP/NF “Ammonio Methacrylate Copolymer Type A”, an ethylmethacrylate/chlorotrimethylammoniumethyl methacrylate copolymer, the copolymer based on acrylic and methacrylic acid esters with a low content of quaternary ammonium groups wherein the molar ratio of the ammonium groups to the remaining neutral (meth)acrylic acid esters is 1:40, the polymer corresponding to USP/NF “Ammonio Methacrylate Copolymer Type B”, a dimethylaminoethylmethacrylate/methylmethacrylate and butylmethacrylate copolymer, a copolymer based on neutral methacrylic acid esters and dimethylaminoethyl methacrylate esters wherein the polymer is cationic in the presence of acids, an ethylacrylate and methylacrylate/ethylmethacrylate and methyl methylacrylate copolymer, the copolymer being a neutral copolymer based on neutral methacrylic acid and acrylic acid esters, ethylcellulose, shellac, zein, and waxes.

150. The formulation of claim 149, wherein said water insoluble polymer comprises ethylcellulose.

151. The formulation of any of the preceding claims wherein said statin is released in the small intestine and/or lower gastrointestinal tract resulting in an increased concentration of at least one active forms in the blood.

152. The formulation of any of the preceding claims wherein said statin is selected from the group comprising simvastatin, beta-hydroxy acid simvastatin, lovastatin, mevastatin, pravastatin, fluvastatin, atorvastatin, pitavastatin, rivastatin and cerivastatin, or pharmaceutically acceptable salts and/or esters thereof.

153. The formulation of any of the preceding claims wherein said statin comprises simvastatin.
